(19) United States
(12) Patent Application Publication
(10) Pub. No.: US 2020/0373017 A1
(43) Pub. Date: Nov. 26, 2020

(54) SYSTEM AND METHOD FOR INTELLIGENCE CROWDSOURCING REINFORCEMENT LEARNING FOR CLINICAL PATHWAY OPTIMIZATION

(71) Applicant: The Trustees of Princeton University, Princeton, NJ (US)

(72) Inventor: Mengdi Wang, Princeton, NJ (US)

(73) Assignee: The Trustees of Princeton University, Princeton, NJ (US)

(21) Appl. No.: 16/882,635

(22) Filed: May 25, 2020

Related U.S. Application Data
(60) Provisional application No. 62/852,624, filed on May 24, 2019.

(51) Int. Cl. G16H 50/30; G16H 10/60; G16H 50/20; G16H 15/00; G16H 20/00; G06Q 40/02

(52) U.S. Cl. CPC G16H 50/30; G16H 10/60; G06Q 40/02; G16H 15/00; G16H 20/00; G16H 50/20

(57) ABSTRACT

Disclosed herein is ICRL4Health, an AI platform technology that provides intelligent predictive and prescriptive decision support for the healthcare industry. It is featured for intelligence crowdsourcing, which is to synthesize the expertise across physicians and doctors to find the best treatment strategy by using data and reinforcement learning technologies. It provides precise and real-time prediction of the best treatment and its outcomes. It provides an end-to-end pipeline for analyzing clinical and claims data, clinical pathway optimization and financial risk management.

### CROSS-REFERENCE TO RELATED APPLICATIONS

This application claims benefit of U.S. Provisional Application No. 62/852,624 filed May 24, 2019, which is hereby incorporated in its entirety by reference.

### TECHNICAL FIELD

The present disclosure is drawn to a system and method for intelligence crowdsourcing reinforcement learning, such as for health pipelines for clinical pathway optimization, healthcare data analytics, and health insurance management. More particularly, disclosed is a system and method for analyzing clinical and/or claims data, predicting outcomes and/or financial costs of clinical pathway, recommending clinical and/or operational decisions, computing optimized clinical pathways, allowing for an analytic tool for healthcare management and insurance analytics.

### BACKGROUND

Driven by the explosion of computing power and big data, the past decade has witnessed tremendous success of artificial intelligence (AI) in many applications, like image recognition, natural language processing, game bot, and speech recognition. Meanwhile, the healthcare industry, with massive volumes of clinical data together with sophisticated devices and recording systems, stands to benefit from this new era of artificial intelligence.

However, artificial intelligence is significantly underdeveloped for clinical decision making in complex situations. Healthcare data are featured for high-dimensionality, heterogeneity, temporal dependency, and irregularity. Machine learning methods can produce good predictions in certain scenarios, but models often lack interpretability, generalizability and global optimization. The healthcare system often involves ineffective services, excessive cost, and uncoordinated cares. Conventional machine learning methods cannot prescribe sequential clinical decision in a systematic way to optimize the entire clinical pathway. There lacks a principled AI-driven approach to optimize the full clinical decision process for complicated medical treatments.

Further, although current approaches may allow one individual decision point to be optimized (for example, an algorithm for determining an optimal dosage(s) of a given drug for a patient with a particular disease), such approaches are incapable of systematically optimizing an entire probabilistic clinical pathway by learning from past data that were collected by one or multiple healthcare providers using suboptimal treatment/decisions, based on individual patient diagnoses, and predicting outcomes based on different treatment pathways.

### BRIEF SUMMARY

The disclosed system and method can be used in the health care field in various ways, all involving the use of intelligent crowdsourcing to allow a reinforcement learning (RL) algorithm within the system to learn from existing data and determine optimal policies, with statistical confidence, that health care workers can then use to inform their decision. Specifically, the method and system are able to predict the conditional distributions of future diagnosis and treatments given any particular time and state of the treatment and prior conditions of the patient. These forecasts can make physicians better informed while making clinical decisions.

A first aspect of the present disclosure is drawn to a system for providing health care providers with optional best-practice care options that they can use to inform their decisions about what care a patient should receive. The system, in operation, includes one or more processors, a database of sequential clinical records, and one or more remote computing devices.

The one or more processors are configured to extract features in a low-dimensional space in order to identify one or more policies. Preferably, the processors are configured with an algorithm that extracts features using a particular program that is specific for the clinical pathway optimization problem, via random feature projection, kernelization, constructing empirical matrix embedding of the unknown transition model, and low-rank approximation. It does so through a series of steps. First, it models a decision-making process as a dynamic state-transition process comprising a series of states and actions, and generate a kernel function that captures the similarity between any two states, based on sequential clinical records, where each record relates to one of a plurality of episodes. The model should be trained using records from multiple patients and multiple physicians. Then, it computes and predicts at least one probabilistic future clinical pathway that a patient would go through, conditioned on the patient's current records. Then, it predicts the clinical pathways for different treatment plans. After that, it determines an empirical transition model and an empirical reward function based on the sequential clinical records, and provides statistical confidence bounds for the optimized empirical transition model and its predictions. The one or more processors then generate a sequence of converging solutions towards an optimal value function and one or more optimal decision policy that maps the optimal decision for each possible state, before obtaining one or more policies based on at least one of the converging solutions, the one or more policies can be used to prescribe actions sequentially along a series of states. Each policy is a mapping from states to actions.

The database contains the sequential clinical records. While this is required during normal operation, the database may be provided separately from the rest of the system. Optionally, the database is provided by a client and loaded/input into the system after the rest of the system has been provided.

The one or more remote computing devices are capable of communicating with the one or more processors. The one or more remote computing devices are configured to receive input from a user relating to a single health-related episode (for example, a clinical record, or several medical claims records). The devices are configured to then transmit the input to the one or more processors. The devices are also configured to graphically or numerically display the one or more policies relating to the health-related episode.

Optionally, the one or more processors may be configured to receive input from the remote computing devices, parse the input, and add one or more records to the database.

Optionally, the policies that are graphically displayed will include at least one policy having the lowest overall cost or a policy where the cost of each claim is below a predetermined threshold. For example, when a doctor is using the system, if the patient does not have health insurance, the patient will need to pay for the treatments out of his or her own pocket. To help with that, one of the policies that are displayed to the doctor might be the policy with the lowest overall cost, or one where the cost of each individual claim falls within some threshold, such as the patient's monthly budget.

Optionally, such as when using medical claims, each record comprises a plurality of attributes, the attributes including a cost, and a code representing a diagnosis, a procedure code, at least one start date, and at least one end date.

While in some cases, records from multiple patients or doctors may be input from a remote computing device, in other instances, the input received from a remote computing device consists of health-related information of a single patient. For example, for a new patient at a medical practice, a medical assistant may enter or load a health-related information for just the new patient into the system.

Optionally, the input from a remote computing device consists essentially of a diagnosis.

Optionally, the one or more processors are configured with automatic feature generation and feature selection based on state aggregation learning and state embedding learning, and tensor decomposition-based state-action representation learning.

Optionally, the outcome can be specified by the client, or, in other cases, the one or more processors are further configured to predict various outcomes, including a financial cost for a current treatment, a financial risk for one or more future treatments, number of future hospital visits, hospitalization duration, health condition at the end of the episode, likelihood of developing complications, other user-specified outcome or a combination thereof.

Optionally, the one or more processors are further configured to estimate leading state and action features and a statistically-optimal reduced-dimension model of state-transition process from trajectorical data via unsupervised spectral state compression.

Optionally, each state is modeled as sₜ=(most recent and/or all related records for a patient up to the current time, number of times the patient has been inpatient up to the current time t).

A second aspect of the present disclosure is drawn to a method for determining optimal policies for a health-related process. The process includes receiving input relating to a single health-related episode, and obtaining one or more policies based on at least one of the converging solutions, the one or more policies can be used to prescribe actions sequentially along a series of states, transmitting the one or more policies to a remote computer, each policy indicating potential actions to be taken currently or in the future, and graphically or numerically displaying the one or more policies to a user of the remote computer.

The method optionally also includes modeling a decision-making process as a dynamic state-transition process comprising a series of states and generate a kernel function that captures the similarity between any two states, based on sequential clinical records, which are typically in a database. The similarity between two states is computed by training from the collection of past data to approximate the divergence between the distributions of future pathways conditioned on the two states. In short, the similarity between two states is measured by the similarity between their future pathways. Then, determining a parameterized and/or nonparameterized empirical transition model and an empirical reward function based on the sequential clinical records and the single health-related episode, and generating a sequence of converging solutions towards an optimal value function.

The method optionally also includes determining a treatment option for a patient based on the displayed one or more policies.

Optionally, the one or more policies are based on an insurance-related factor.

The method optionally also includes parsing input received and adding at least one record to the database.

Optionally, the one or more policies may include a policy having the lowest overall cost or a policy having a cost below a predetermined threshold.

Optionally, each record comprises a plurality of attributes, the attributes including a cost, and a code representing a diagnosis, a procedure code, at least one start date, and at least one end date.

Optionally, the input consists of health-related information of a single patient, and may consist essentially of a diagnosis.

Optionally, the one or more processors are configured with automatic feature generation and feature selection based on state aggregation learning and state embedding learning.

Optionally, the one or more processors are further configured to predict various outcomes, including a financial cost for a current treatment, a financial risk for one or more future treatments, number of future hospital visits, hospitalization duration, health condition at the end of the episode, other user-specified outcome or a combination thereof.

### BRIEF DESCRIPTION OF DRAWINGS

FIG. 1 is a flowchart of one embodiment of a disclosed method.

FIG. 2A is a graph illustrating in-sample and out-of-sample performances of the optimized policy with varying degrees of model complexity, as well as their 95% confidence intervals.

FIG. 2B is a graph illustrating the in-sample and out-of-sample performances of excessive cost premium per episode of the best trained policy based on a particular claims data set about knee replacement.

FIG. 3A is a bar plot showing an example of a graphical display showing, for each day of a full clinical pathway, the probability distribution of a patient's state over, e.g., the 138 diagnosis categories, where the x-axis corresponds to the day of treatment, and Y-axis corresponds to 138 diagnosis categories (each has a unique color) based on a particular claims data set about knee replacement.

FIG. 3B is a bar plot showing an example of a graphical display giving forecasts of the future pathway from day 25, conditioned on the event that the current diagnosis belongs to the osteoarthritis category.

FIG. 4 is an alternative embodiment of a disclosed method.

FIG. 5 is a simplified block diagram of one embodiment of a disclosed system.

FIG. 6 is an illustration of a tensor-inspired state and action embedding method.

FIG. 7A is an illustration depicting Block MDP (aka hard aggregation).

FIG. 7B is an illustration depicting Latent-state-action MDP (aka soft aggregation).

FIG. 8 is a visual flowchart illustrating the state embedding learning process for analyzing random walk data (a simplified state-transition process) to find critical meta-states.

FIG. 9 is a diagram of the state embedding method applied to the trajectory of a game using the composition of deep neural network and Gaussian kernel for low-rank approximation.

FIGS. 10A and 10B are visualizations of game states before (10A) and after (10B) embedding in t-SNE plots. The raw data is a time series of 512 dimensions, which generated by the last hidden layer by the DQN while it is playing Demon Attack. State embeddings are computed from the raw time series using a Gaussian kernel with 200 random Fourier features. Game states are colored by the “value” of the state as predicted by the DQN. The markers (diamonds 1001, 1002), circles (1003, 1004), and triangles (1005, 1006) identify pairs of game states that share similar transition distributions before and after embedding. FIGS. 10A and 10B show that the state embedding method can identify states based on their similarity in their future dynamics.

FIG. 11 is an illustration of a model of a clinical pathway as a state-transition process.

### DETAILED DESCRIPTION

The disclosed system and method can be used to allow health care professionals to determine a treatment pathway in the health care field, based on the RL algorithm's optimal policies. In some embodiments, a doctor treating a patient can use the system to better understand the impact of different care options, which they can then use to inform their decisions about what care a patient should receive or what treatment plan might be needed. In other embodiments, a hospital might use the system to determine which of their physicians might be best for particular segments of a treatment plan. In still other embodiments, a doctor, hospital, or insurance company may use the system to predict the performance (such as cost and healthcare outcome) of a new treatment policy that has not been implemented before, based on existing data generated by following current treatment policies.

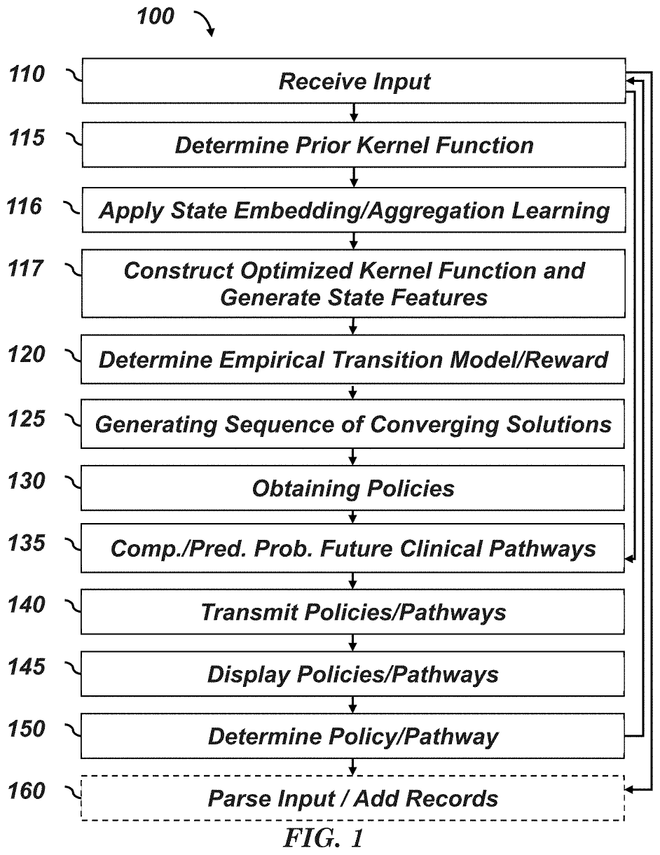

Referring to FIG. 1, the disclosed method 100 can be envisioned as a machine learning procedure for providing options that a health care professional can use to determine a path forward for a patient's treatment.

In FIG. 1, the first step of the method 100 includes receiving 110 some input from a user. Typically, the user will be a health care professional, and may be in an office or hospital. That input will generally relate to a single health-related episode, and could be, for example, a clinical record, or medical claims records. While in some cases, records from multiple patients or doctors may be input from a remote computing device, in other instances, the input received from a remote computing device consists of health-related information of a single patient. For example, for a new patient at a medical practice, a medical assistant may enter or load a health-related information for just the new patient into the system. In some embodiments, the input consists essentially of one or more diagnoses. This input information is then used to help inform what the best policies are.

Note that while FIG. 1 shows this step as occurring before all other steps, the input may optionally be received at any time prior to the computing and predicting 135 of probabilistic future clinical pathways. It is envisioned that there are typically two types of input. A first type of input is a large amount of data (e.g., a database or data set) for training the machine learning algorithm. Given such input, the algorithm will compute the state feature/kernel function and the optimized low-order empirical transition model. The second type of input is records (clinical records and/or medical claims) about a new patient. In this part, the algorithm will generate predictions and optimal policies for this single patient.

In FIG. 1, the second step is modeling a decision-making process as a probabilistic dynamic state-transition process comprising a series of states and actions, and generate a kernel function that captures the similarity between any two states, which is an approximate divergence function between their future distributions of pathways (it can be viewed as a restricted diffusion distance customized to the specific probabilistic process and computed from data), based on sequential clinical records.

Typically, the sequential clinical records will contain records from multiple patients (preferably at least 100 patients, more preferably at least 1,000 patients who went through identical or highly similar treatment episodes) and multiple physicians (preferably at least 25 physicians, more preferably at least 100 physicians). Preferably, these records are contained in a database.

While what is included in each record may vary, in preferred embodiments, each record comprises a plurality of attributes, the attributes including a cost, and a code representing a diagnosis, a procedure code, at least one start date, and at least one end date. Other fields, including patient names or codes, physician names or code, a code representing a particular episode, insurance-related codes, etc., may optionally be included in some embodiments.

The modelling is typically done by determining 115 a prior kernel function over the state space, by using deep neural network or other user-specified function. Then, applying 116 state embedding/aggregation learning to find an optimal low-rank approximation of the empirical transition model and state features. The state features are then used to construct 117 an optimized kernel function that measures the similarity between medical states in terms of the divergence between their transition distributions to future states.

The method also includes determining 120 an empirical transition model and an empirical reward function based on the sequential clinical records and providing statistical confidence bounds for the estimated transition model and its predictions. Said differently, the optimized state feature/kernel functions are used to determine the empirical transition model and the reward function, in either parametric and nonparametric representations. From there, the method involves generating 125 a sequence of converging solutions towards an optimal value function and an optimal decision policy that maps the optimal decision for each possible state. Then, the method includes obtaining 130 one or more policies based on at least one of the converging solutions, the one or more policies can be used to prescribe actions sequentially along a series of states. In some embodiments, the one or more policies are based on an insurance-related factor. In some embodiments, the one or more policies includes a policy having the lowest overall cost or a policy where each claim has a cost below a predetermined threshold.

The method then involves computing and predicting 135 at least one probabilistic future clinical pathway that a patient would go through conditioned on the patient's current records, based on at least one policy. In some embodiments, the method involves predicting the clinical pathways for two or more different treatment plans.

In some embodiments, the method also includes using either one or more optimized policies obtained in the previous step or any user-specified policy to compute and predict the posterior probability distribution of two or more most likely clinical pathways that a patient would go through conditioned on the patient's current records, and/or using either one or more optimized policies obtained in the previous step or any user-specified policy to compute and predict the overall financial cost or other specified outcome and the estimated value's statistical confidence region.

The following example is one approach that can be taken for these steps.

### Example 1—Knee Replacements

Knee replacement is a surgical procedure using metal and plastic parts to resurface damaged knees. An episode of knee replacement treatment takes approximately three months. During this process, a patient goes through a pathway from onset to surgery, rehabilitation and recovery. According to the Centers for Medicare and Medicaid Services (CMS) hip and knee replacements are the most common inpatient surgery in the United States. Around 700,000 knee replacement procedures take place every year. The average cost per episode ranges from $16,500 to $33,000 across different geographical areas. Patients who receive the knee replacement treatments often have to go through a long recovery time. Knee replacement treatments are costly for the patients and the Medicare, and the quality of treatments vary largely among healthcare providers.

Beginning on Apr. 1, 2016, the Centers for Medicare and Medicaid Services (CMS) started a new payment model, called the Comprehensive Care for Joint Replacement (CJR) model, to reduce excessive costs and support better care for patients who are undergoing elective hip and knee replacement surgeries. The Cat model will run for five years in 67 geographic areas. According to the CJR, more than 800 hospitals are required to keep the knee replacement cost below $25,565 for each episode, and they will face a financial penalty for exceeding this threshold. According to some available data, after two years since it was implemented, the CJR model achieved a cost reduction of $812 per episode, which is a reduction of 3.1%. Motivated by the pressing needs for cost reduction and better care, this platform was designed to optimize the clinical pathway of knee replacements, by leveraging the AI technology to learn the best clinical decisions from data.

The starting point is a data set consisting full episodes of claims records from 212 successful past treatments. The records are collected from 37 care providers who are all experts in treating knee replacements. The clinical pathway optimization was viewed as an intelligence crowdsourcing problem. Care providers may have different strengths—some of them might make better decisions at certain stages of the process. The goal is to “imitate” the best expert at different “states” of the process.

The data set contains claims records from 37 physicians and 205 unique beneficiaries (patients). It contains 212 episodes, 5946 claims and 9254 entries. Each episode is a full claims record for a patient' pathway from onset to rehabilitation and recovery. Each episode involves one physician, one beneficiary, a total cost, an episode start date, an episode end date, and a sequence of claims. Table 1 gives a snapshot of several lines in the data set. We have masked the sensitive information to protect the patient's identity.

Attributes: Each entry contains the following attributes: episode id, beneficiary id, episode start date, episode end date, episode total cost, physician id, claim id, claim start date, claim end date, claim cost, procedure code, procedure category, diagnosis code, diagnosis category.

Date: Each episode has one “episode start date” and one “episode end date”, and each claim has one “claim start date” and one “claim end date”. Most claims start and end on the same day. But still, there exists some claims whose “claim start date” and “claim end date” are different.

Claim: Each claim consists of diagnoses, procedures, a start date, an end date and a cost of the claim. There are 597 different procedures, 81 procedure categories, 542 different diagnoses, and 138 diagnosis categories in this data set. Almost all claims involves only one diagnosis and/or only one procedure. For example, see line 6 in FIG. 1, where “NA” means the corresponding procedure or diagnosis does not exist. Some claims have more than one entries.

Beneficiaries and Physicians: There are 37 physicians and 205 beneficiaries in this data set. Each beneficiary is attributed to a unique physician.

Duration and Cost: The duration of episodes are all around 100 days, because knee replacement is a standardized and well-scheduled process. The number of claims per episode ranges from 8 to 56.

For confidentiality purposes, no additional information about the data set can be disclosed.

TABLE 1
First Eight Lines of Knee Replacement Data, Select Columns

| Episode | Claim | Procedure | Procedure | Diagnosis | Diagnosis |
|---|---|---|---|---|---|
| Total Cost | Cost | Code | Category | Code | Category |
| 18012.5 | 287.16 | 1402 | 232 | 71596 | 203 |
| 18012.5 | 1160.14 | 27447 | 152 | 71596 | 203 |
| 18012.5 | 7.19 | 73560 | 226 | V5481 | 257 |
| 18012.5 | 185.62 | 27447 | 152 | 71596 | 203 |
| 18012.5 | 12014.14 | NA | NA | 71596 | 203 |
| 18012.5 | 93.62 | E0163 | 243 | NA | NA |
| 18012.5 | 42.19 | E0143 | 243 | NA | NA |
| 18012.5 | 2611.40 | 1AGP1 | NA | V571 | 254 |

The pipeline provides forecast of future pathway at any intermediate stage under the optimal policy. The pipeline employs a dimension reduction technique to automatically aggregate histories and diagnosis in a way to maximally preserve the quality of the solution. It also uses cross validation to find the most robust solution even if data is limited, as well as to provide confidence bound of the predicted performance. The optimized policy in this example reduces the average cost per episode by $1,000, which is approximately 7%. For the portion of the cost above the repayment threshold, the optimized policy reduces the average excessive premium by $500, which is approximately 33%.

If successfully validated and implemented, the optimized treatment policy is projected to result in at least $700 million in savings each year in the US. The proposed approach provides a prototype for machine learning-based clinical pathway optimization. It is believed it will apply to a broader scope of treatments and can significantly improve the outcome of care, leading to cost reduction and better patient experience.

A reinforcement learning-based approach was developed to learn the optimal policy from episodic claims. Reinforcement learning is a branch of artificial intelligence. It is well-suited for the sequential, uncertain and variable nature of clinical decision making. By modeling the knee replacement episode as a Markov decision process, a reinforcement learning pipeline to find the optimal treatment decision at every possible state, that takes its long-term effect into account, was developed.

The platform includes a reinforcement learning-based pipeline to analyze episodic claims records and synthesize expertise of multiple physicians into an optimal treatment policy.

Reinforcement learning (RL) is an area of machine learning for solving sequential decision-making problems from data or experiment. RL has achieved phenomenal successes in many fields. Compared with conventional machine learning, RL focuses on learning and acting in a dynamic environment, which is powerful to model and recommend sequential decisions to be made dynamically in clinical treatments.

There have been several attempts to apply RL in healthcare scenarios. For sepsis treatment optimization, one group proposed a continuous-state framework to generate optimal treatment policies with deep reinforcement learning, and another developed a mixture-of-experts framework by combining neighbor-based algorithms and deep RL. One group modeled heparin dosing as a Partially Observed Markov Decision Process (POMDP), and designed a deep RL algorithm to learn individualized heparin dosing policy. Another group used off-policy reinforcement learning to improve the dosing policy in ICU. Still others used the kernel-based regression and dynamic programming to improve therapy selection for treating HIV. In these applications, the decision is often the dosage, which is a single-dimensional variable, or the decision is restricted to a small number of choices to reduce the problem's complexity.

For knee replacement treatment, there exist studies where machine learning approaches are used for prediction or classification. However, these studies did not consider the optimization of clinical decision process.

This example appears to be the first one to systematically optimize the knee replacement treatment. The proposed approach uses ideas including kernel reinforcement learning, state aggregation and dimension reduction. Kernel-based reinforcement learning was proposed in 2002. A kernel function is needed to measure the similarity between states, based on which one can approximately solve high-dimensional Markov decision problems. It is shown that kernel-based RL produces a convergent solution that also achieves statistically consistency. Such a model has been studied and extended in a number of works. A critical component of this example is to learn the right kernel automatically from data. The kernel function was designed by using unsupervised spectral state compression and aggregation learning. Further, there is a method to estimate the optimal partition of state space from trajectoric data to maximally preserve the transition dynamics in the data set. In this way, this example is able to remedy the curse of dimensionality of high-dimensional RL by leveraging features that are learned from data.

This approach provides a novel end-to-end pipeline for clinical pathway optimization from clinical data. It allows one to learn from records of a group of medical experts to achieve intelligence crowdsourcing by using statistical dimension reduction and reinforcement learning. The outcome is a full decision policy with robustness guarantee, together with a predictive model to forecast a patient's clinical pathway beginning from any intermediate state of the process.

### Model and Formulation

The clinical decision-making process of knee replacement was modeled as an infinite-horizon Markov decision process (MDP) with an absorbing terminal state. MDP models a dynamic state-transition process, where the state sₜ at time t evolves to a future state sₜ₊₁ according to a transition law under the intervention of a decision maker. An instance of MDP can be described by a tuple M=(S, A, P, C), where S is set of all possible states, A is set of all possible actions, P is a law of transition which is not explicitly known to the decision maker, and C:S×A→R is a state-transition reward function.

In the case of knee replacement, each claim is modeled as a time step. The knee replacement process may last for an indefinitely number of time steps. “Recovery” is modeled as an absorbing terminal state, at which no future transition or cost will be generated. According to the data set, all episodes terminate at the recovery state. In a given episode, ideally speaking, a state sₜ is a collection of claims up to the time step t, and an action aₜ is picked from all possible prescriptions. At time t, a physician examines the current state sₜ∈S of a patient, chooses an action aₜ∈A according to his/her own expertise and then the patient moves to the next state sₜ₊₁ according to some probability P(sₜ, aₜ , sₜ₊₁). Each claim may generate a cost C(sₜ, aₜ).

As used herein, the term “cost” is intended to include any criteria which may be used by the model for optimization, including, e.g., monetary costs, time and other user-specified health-related outcome.

The treatment policy is modeled as a function that maps a state (claims history) to an action (a prescription), which is denoted by Π:S→A. The goal is to find an optimal policy to minimize the expected cumulated cost per episode, i.e., to solve the optimization problem

$${min}_{π} {𝔼}^{π} [ {\sum }_{t = 0}^{∞} C ( {s}_{t} , {a}_{t} ) ] ,$$

where EΠ is taken over the entire pathway (s₁, a₁, s₂, a₂, . . . ) on which actions are chosen according to the policy Π. Solving this ideal optimization problem precisely is intractable because the state-to-state transition probabilities {P (s, a, s′)} are unknown. Given 138 different diagnosis categories and around 30 claims per episode, the number of possible states |S| can be as large as 138³⁰≈10⁶⁴. The number of possible actions is 597 according to the data set. Such huge numbers of states and actions make reinforcement learning intractable.

Therefore, the policy optimization problem can be formulated in the spirit of intelligence crowdsourcing. The state is modeled as:

sₜ=(most recent and/or all related records for a patient up to time t, number of times one has been inpatient up to time t),

which is still sufficient to capture most of the information needed for making decisions.

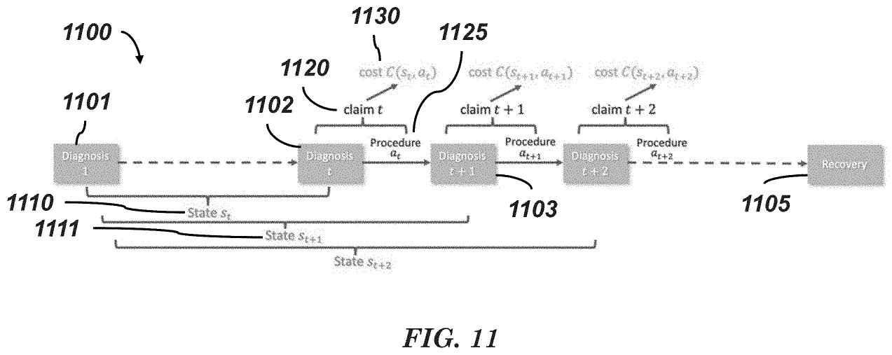

An illustration of this can be seen in FIG. 11. There, the model 1100 of a clinical pathway as a state-transition process is shown. One can see a series of diagnoses 1101, 1102, 1103 that eventually leads to recovery 1105. For each diagnosis, there may be an associated claim 1120, which may have a corresponding action/procedure 1125 and cost 1130 associated with the claim. At any time t, the state sₜ 1110, 1111, can be seen as the diagnoses, claims, procedures, and costs up to time t. So in FIG. 11, moving from state sₜ 1110 to sₜ₊₁ 1111, it can be seen that state sₜ₊₁ includes all records from sₜ, plus records relating to claim 1120, procedure 1125, cost 1130, and diagnosis 1103.

This still yields a large state space. Further dimension reduction is desirable. The physicians were split randomly into multiple groups 1, . . . , J and it was made sure that these groups have identical average cost per episode. Suppose the current state is sₜ, the action aₜ∈A:={1, . . . , J} can be modeled to be: First pick a group of physicians from 1, . . . , J, then pick one doctor from the group uniformly at random, finally imitate the prescription of this physician at the current state sₜ. Thus, the resulting state-to-prescription policy is a randomized policy. Since A has been reduced to be a small finite set, the action complexity has been reduced significantly. More importantly, this model can maximally leverage the physicians' expertise from their past experiences.

### State Compression and Kernel Construction

To find a robust policy from limited noisy data, dimension reduction tools are needed to identify latent features of the knee replacement process and further reduce the model complexity.

An unsupervised spectral state compression method was employed. The goal is to estimate leading features of a state-transition process (X₁, X₂, . . . , Xₜ) from trajectorical data. It is motivated by common latent structures of state-transition system, such that the transition kernel often admits a low-rank decomposition:

$$P ( {X}^{′} \vert X ) \approx {\sum }_{z} P ( {X}^{′} \vert Z = z ) P ( Z = z \vert X ) .$$

The low-rank nature makes it possible to estimate the left and right singular space of the unknown transition matrix P from data.

The first step is to compute the empirical transition frequency matrix F∈R^{|S|×|S|} from the data set. All the claims are ordered according to claim id in each episode. Every two consecutive claims gives a state-transition pair (s, s′). The entry F_{ss′} is computed to be the frequency for (s, s′). to appear in the entire data set. After obtaining F, one can normalize it into an empirical transition matrix P, such that each row is nonnegative and sums to 1. Then we apply singular value decomposition (SVD) to the transition matrix P=U Σ V and get the right top k singular vectors of P, where k is a tuning parameter. It gives a matrix \hat{V}∈R^{|S|×k}. \hat{V} are referred to as Markov features that are representative of the leading structures of the unknown transition matrix.

The optimal partition of state space S=S₁∪S₂∪ . . . ∪Sₖ is computed such that P(⋅|s)≈P(⋅|s) if s, s′∈Sⱼ for some j∈[k]. In this way, one can aggregate states into clusters and find one best action for all states belonging to the same cluster. The partition can be computed by solving the optimization problem and using the estimated \hat{V}:

$${min}_{{𝒮}_{1} , … , {𝒮}_{k}} {min}_{{υ}_{1} {υ}_{2} , … , {υ}_{k} ∈ {ℝ}^{k}} {\sum }_{m = 1}^{k} {\sum }_{s ∈ {𝒮}_{m}} {‖ {( \hat{V} )}_{[ s , : ]} - {υ}_{m} ‖}_{2}^{2} .$$

In the example, the preceding problem was solved by applying the k-means method with 100 random initializations and choosing the best result.

Now there are estimated Markov features \hat{V}, and an optimal partition by using spectral state compression.

One can define a kernel function for states by K(s₁,s₂)=\hat{V}_{[s}_{_{1,:}}_{]}\hat{V}_{[s}_{_{2,:}}_{]}. Alternatively, one could, e.g., use the estimated partition and define the kernel function by K(s₁, s₂)=1 if s₁, s₂ belongs to the same block and K (s₁, s₂)=0 otherwise.

### Kernel-Based Model Estimation

So far, the example has obtained a kernel function K(⋅, ⋅) that captures the similarity between any two states. It is a non-negative mapping defined as K: S×S→R⁺. Now one can use the kernel function to construct an empirical MDP model \hat{M}=(S, A, \hat{P}, Ĉ) from the data, where S, A are the same state space and action space as in the original MDP M.

Let D={(sₘ,aₘ,cₘ, s′ₘ)|m=1,2, . . . , n} be the set of sample transition quadruples (state, action, cost, next state). Each sample comes from two consecutive claims, and n is the total number of such samples. Now one can estimate the state-transition probability matrix \hat{P} as

$$\hat{P} ( s , a , {s}^{′} ) = \frac{{\sum }_{\{ m \vert {a}_{m} = a \}} K ( s , {s}_{m} ) K ( {s}^{′} , {s}_{m}^{′} )}{{\sum }_{\{ m \vert {a}_{m} = a \}} K ( s , {s}_{m} )} \qquad ( 1 )$$

for any s, s′∈S and a∈A, as long as s is not the terminal recovery state. If s is the terminal state, the model will always let \hat{P}(s, a, s)=1 and \hat{P}(s, a, s′)=0 for all a∈A and s′≠s.

One can estimate the cost function Ĉ using

$$\hat{C} ( s , a ) = \frac{{\sum }_{\{ m \vert {a}_{m} = a \}} K ( s , {s}_{m} ) K ( s , {s}_{m} ) {c}_{m}}{{\sum }_{\{ m \vert {a}_{m} = a \}} K ( s , {s}_{m} )} \qquad ( 2 )$$

for any s∈S that is not the terminal state and any a∈A. For the terminal state, the example lets Ĉ(s, a)=0 for all a.

### Computing the Optimal Policy

By using the appropriate kernel function, the example has computed the empirical transition model (1) and empirical reward function (2) from claims data. The example has obtained an empirical instance of \hat{M}=(S, A, \hat{P}, Ĉ), which is an approximation to the unknown true MDP M.

Finally, one can solve this empirical MDP problem \hat{M} by using the value iteration method. The value iteration makes the following iterative updates on a value vector v∈R^{|S|}:

$$υ ( s ) ← {max}_{a ∈ A} [ \hat{C} ( s , a ) + {\sum }_{{s}^{′} ∈ 𝒮} \hat{P} ( s , a , {s}^{′} ) \cdot υ ( s ) ] , ∀ s ∈ 𝒮 ,$$

with the terminal condition v(terminal)=0. The value iteration is known to provide a sequence of solutions converging to the optimal value function v*∈R^{|S|}. Then one obtains the optimal policy Π*:

$${π}^{*} ( s ) ← {arg max}_{a ∈ A} [ \hat{C} ( s , a ) + {\sum }_{{s}^{′} ∈ 𝒮} \hat{P} ( s , a , {s}^{′} ) \cdot {υ}^{*} ( s ) ] .$$

The computed Π* prescribes a physician group to follow for each state. The final treatment policy is a set of conditional distributions over prescriptions, which are estimated from the data set following Π*.

### Projected Performances and Cross Validation

In the example, the diagnosis categories were divided into k clusters by using the spectral state clustering method described above, for k=2, . . . , 9. The partition map is further used to build a kernel function for the state space S. One can view k, the rank parameter in state compression, as a measure of the model complexity. It is a tuning parameter that controls the tradeoff between model reduction error and overfitting error.

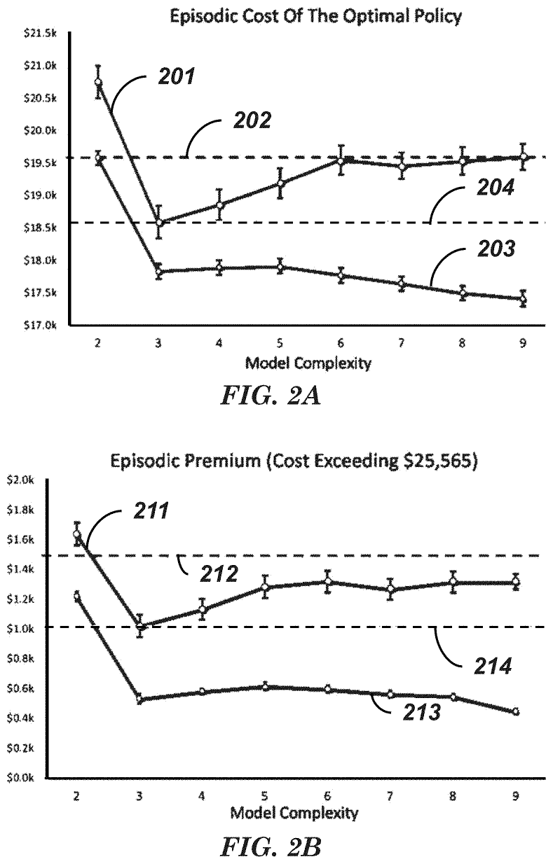

One can conduct both in-sample and out-of-sample experiments. In in-sample experiments, the treatment policy is trained to minimize cost over the entire data set, and test its performance on the same raw data. In out-of-sample experiments, one can use 4-fold cross validation. The raw data set is split into 4 equal-size segments randomly, and the optimal policy is computed using three out of the four segments. Its performance is tested on the remaining data. Each experiment in this example was repeated randomly for 2,000 times and for all values of k. Further, note that according to some sources, hospitals have to keep their costs below $25,565 or they will face a financial charge. Here, the episodic excessive cost premium is defined as episodic premium=max{0, episode cost−25565}. The results are given in FIGS. 2A and 2B. FIGS. 2A and 2B are graphs illustrating the in-sample 203 vs out-of-sample 201 performance of the learned optimal policy. FIG. 2A illustrates the in-sample and out-of-sample performances of the optimized policy with varying degrees of model complexity (various values of k). The baseline 202 is the average episodic cost in the raw data set. These curves suggest that the model tends to overfit the data as k>3. The optimized policy 204 is projected to achieve a cost reduction of $1,000 per episode. FIG. 2B illustrates the in-sample 213 and out-of-sample 211 performances of excessive cost premium per episode (cost above $25,565 per episode) of the best trained policy. The baseline 212 is the average episodic premium in the raw data set. The best out-of-sample performance 214, is achieved when the model complexity is k=3. In this case, the optimized policy is projected to reduce the excessive premium per episode by approximately $500 or 33% on average.

The results of FIGS. 2A and 2B are consistent with typical in-sample vs out-of-sample error curves for training machine learning models. It is expected that the in-sample error decreases monotonically as the model complexity k increases, however, over-fitting would occur when k is too large. The out-of-sample error curves show that over-fitting occurs when k>3, therefore one would pick k=3 to be the best model complexity for this data set. One can take the out-of-sample performance at k=3 as the predicted performance of the optimized policy. In this case, the average cost per episode reduces by 7 percent and the average cost premium per episode reduces by 33 percent, or approximately $500.

### Cost Distribution and Tail Improvement

One can compare the histograms of episodic cost over the raw data and after optimization. The after-optimization cost distribution is obtained by simulation. The percentages of episodes costing over $30k are reduced significantly.

Such a reduction mitigates the financial risk faced by health-care providers, as they are penalized for costs over $25,565 according to CMS.

In this example, a histogram of the results would show a tail improvement that is significant for reasons beyond financial risk. It is well known that excessive medical cost in medical treatment is highly correlated to unsuccessful cases, for example post-surgery complications often lead to extra surgeries, higher costs and traumatic discomforts. Therefore, the tail cost reduction indicated by the results suggests that the optimized clinical policy may reduce unnecessary medical complications and extra inpatient surgeries. Therefore, the proposed pipeline not only reduces financial costs but also improves the quality of care.

### Clinical Pathway Forecast Using the Optimal Policy

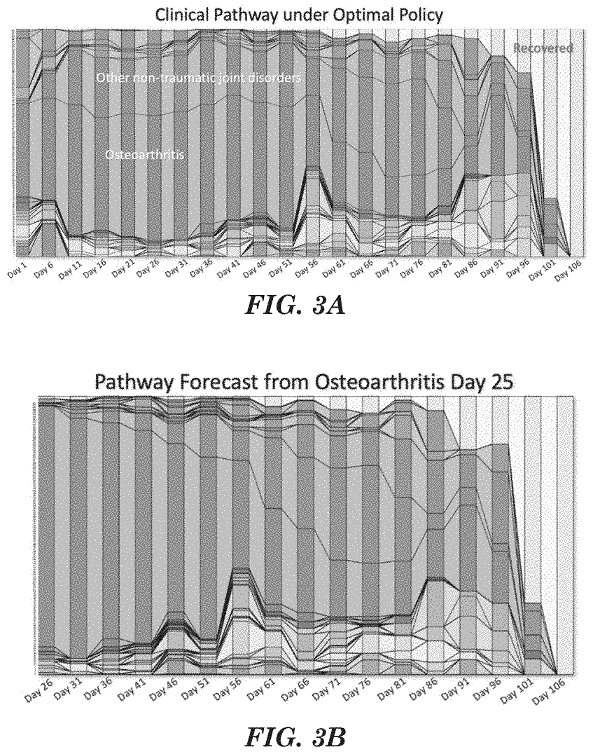

Now that there is one or more estimated best clinical policies based on the data, the model allows one to predict the entire clinical pathway of a typical knee replacement treatment. One can simulate the knee replacement process under the optimized policy based on existing claim episodes. This generates 10,000 sample trajectories. Then, based on the sample trajectories, one can compute the empirical distribution of diagnosis categories per each day. A graphical representation of this can be seen in FIG. 3A.

The model can also forecast the future clinical pathway of a patient from any given time and given state during the knee replacement episode. As a simple example, one could forecast the pathway from day 25 conditioned on the event that the current diagnosis belongs to the category of osteoarthritis. One graphical representation of this forecasted pathway can be seen in FIG. 3B. Other examples include forecasts of pathways from day 50 or 75, conditioned on the event that the current diagnosis belongs to the category of osteoarthritis, or forecasts of pathways from day 50, conditioned on the event that the current diagnosis belongs to the category of other non-traumatic joint disorders. One can see that the model is able to predict the conditional distributions of future diagnosis and treatments given any particular time and state of the treatment. These forecasts can make physicians better informed while making clinical decisions. In some embodiments, the model also predicts a financial cost for a current treatment, a financial risk for one or more future treatments, number of future hospital visits, hospitalization duration, health condition at the end of episode, other user-specified metrics, or a combination thereof.

Referring back to FIG. 1, the next step in the model is to transmit 140 the policies and/or pathways (and optionally other forecasted information, such as financial risk for one or more future treatments, etc.), typically to a remote computing device, which may optionally be done via a wired or wireless connection. The remote computing device then graphically or numerically displays 145 the policies and/or pathways to the user.

The method may optionally also includes determining 150 a treatment option for a patient based on the displayed one or more policies and/or pathways. For example, a physician for a sports team could use the method to determine how to help a player on the sports team recover fastest, even if it costs slightly more money than another policy.

Typically, this determination step is determined by one or more users, alone or in combination with other people (such as the patient, or others in the health care field). The user is typically a health care professional, such as a physician, nurse, surgeon, or administrator, or someone who works in a health care-related field, such as insurance.

The method may also optionally include parsing the received input and adding a record to the database. For example, if the input included multiple records, the method may include parsing the received records and inserting information from the records into appropriate fields in a database containing sequential records.

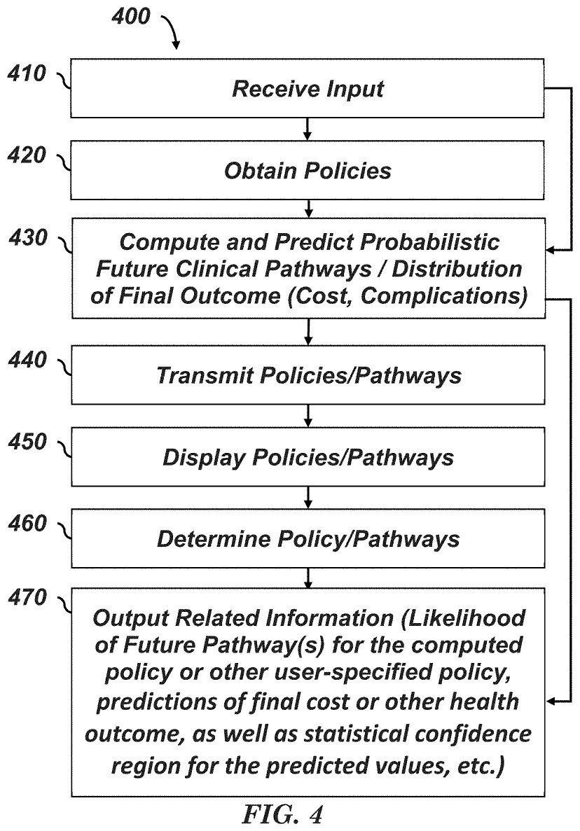

An alternative embodiment of a method is shown in FIG. 4. There, the method 400 first involves receiving input 410 from a user. The method 400 then involves obtaining 420 one or more policies from a trained model. The model is, as discussed previously, a reinforcement learning model trained using crowdsourced intelligence. The policies are then used to compute and predict 430 probabilistic future clinical pathways that a patient would go through conditioned on the patient's current records. The policies and/or pathways are then transmitted 440 to a remote computing device, where the policy and/or pathway is displayed 450 to a user. At that time, a user may optionally determine 460 which policy and/or pathway will be followed moving forward.

In some embodiments, the method may also involve computing and outputting information related to the clinical pathway. For example, in some embodiments, at some point in time, the method also involves outputting 470 related information, such as the likelihood of future pathway(s) for the computer policy or other user-specified policy, or predictions of final cost or other health outcome, as well as statistical confidence region for the prediction values. This can be done, by, e.g., using either one or more optimized policies obtained in the previous step or any user-specified policy to compute and predict the posterior probability distribution of two or more most likely clinical pathways that a patient would go through conditioned on the patient's current records, and/or using either one or more optimized policies obtained in the previous step or any user-specified policy to compute and predict the overall financial cost or other specified outcome and the estimated value's statistical confidence region.

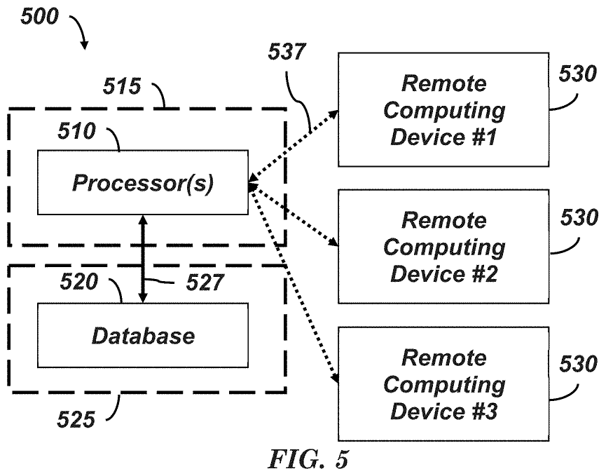

Various systems are envisioned the incorporate the use of this method. Referring to FIG. 5, one embodiment of a system 500, in normal operation, utilizes one or more processors 510, a database 520 of sequential clinical records, and one or more remote computing devices 530.

The one or more processors 510 may be co-located or may be in one or more locations 515. For example, in a cloud-based system, one or more processors may be in different countries. The one or more processors 510 are configured to electronically communicate 127 with database 520.

The database 520 may be co-located with the one or more processors 510 or may be in one or more separate locations 525. Database 520 may electronically communicate with the one or more processors 510 via any mode of communication, including any known wired or wireless form of communication, as appropriate.

The remote computing devices 530 include any type of known computing device. Preferably, the devices are desktop or laptop computers, smartphones, or mobile tablets, and may be in multiple remote locations. In some embodiments, each of the remote computing devices 130 is located within a hospital. Remote computing devices 130 are configured to electronically communicate with the one or more processors 110 via any mode of communication, including any known wired or wireless form of communication, as appropriate.

The one or more processors 110 may be configured to receive input from a user, where the input may be a single health-related episode, such as a clinical record, or medical claims records. The input may also be one or more diagnoses.

The one or more processors 110 may be configured to compute and predict at least one probabilistic future clinical pathway that a patient would go through, conditioned on the patient's current records, using at least one policy received from a trained model.

The one or more processors 110 may also be configured to extract features in a low-dimensional space in order to identify one or more policies. It does so through a series of steps. First, it models a decision-making process as a dynamic state-transition process comprising a series of states and actions, and generate a kernel function that captures the similarity between any two states, based on sequential clinical records, where each record relates to one of a plurality of episodes. Optionally, each state is modeled as sₜ=(most recent and/or all related records for a patient up to time t, number of times the patient has been inpatient up to time t). The model should be trained using records from multiple patients and multiple physicians. After that, it determines an empirical transition model and an empirical reward function based on the sequential clinical records, and provides statistical confidence bounds for the optimized empirical transition model and its predictions. The one or more processors then generate a sequence of converging solutions towards an optimal value function and an optimal decision policy that maps the optimal decision for each possible state, before obtaining one or more policies based on at least one of the converging solutions, the one or more policies can be used to prescribe actions sequentially along a series of states. Then, it can compute and predict at least one probabilistic future clinical pathway that a patient would go through, conditioned on the patient's current records.

The one or more processors may also be configured to then transmit the policy and/or pathway to one or more remote computing devices.

The one or more processors may be configured to receive input from the remote computing devices, parse the input, and add one or more records to the database based on the input.

Preferably, the one or more processors are further configured with automatic feature generation and feature selection based on state aggregation learning and state embedding learning, and tensor decomposition-based state-action representation learning. See, e.g., Sun, et al., “Learning low-dimensional state embeddings and metastable clusters from time series data”, in Advances in Neural Information Processing Systems 2019 (pp. 4563-4572); Zhang, et g, “Spectral State Compression of Markov Processes” IEEE Transactions on Information Theory, 66 (4): 3202-3231, 2019; Duan, et al., “State Aggregation Learning from Markov Transition Data” NeurIPS, 2019, all of which are incorporated by reference herein in its entirety.

Preferably, the one or more processors are further configured to predict various outcomes, including a financial cost for a current treatment, a financial risk for one or more future treatments, number of future hospital visits, hospitalization duration, health condition at the end of the episode, other user-specified outcomes or a combination thereof.

Preferably, the one or more processors are further configured to estimate leading features and a statistically-optimal reduced-dimension model of state-transition process from trajectorical data via unsupervised spectral state compression.

The database contains the sequential clinical records. While this is required during normal operation, the database may be provided separately from the rest of the system. Optionally, the database is provided by a client and loaded/input into the system after the rest of the system has been provided.

The one or more remote computing devices are capable of communicating with the one or more processors. The one or more remote computing devices are configured to receive input from a user relating to a single health-related episode (for example, a clinical record, or several medical claims records). The devices are configured to then transmit the input to the one or more processors. The devices are also configured to graphically or numerically display the one or more policies relating to the health-related episode.

Optionally, the policies that are graphically displayed will include at least one policy having the lowest overall cost or a policy where the cost of each claim is below a predetermined threshold. For example, when a doctor is using the system, if the patient does not have health insurance, the patient will need to pay for the treatments out of his or her own pocket. To help with that, one of the policies that are displayed to the doctor might be the policy with the lowest overall cost, or one where the cost of each individual claim falls within some threshold, such as the patient's monthly budget.

### Example 2

One key step to reduce the dimension of the problem before computing the empirical transition model or the optimal policy involves “state embedding.” FIG. 9A is an illustration related to state embedding (or state aggregation). The state embedding method is compatible with deep neural networks. It further uses random feature projection and low-rank compression, and outputs a further optimized low-dimensional representation of states. The output embedding (e.g., Φ) determines (1) the kernel function measuring similarity between states, (2) parametrization of the empirical transition model and (3) policy optimization problem. The low-dimensionality of embeddings guarantees the statistical confidence of the optimal strategy learned by the reinforcement learning algorithm.

FIG. 9A is a visual flowchart illustrating a simplified state embedding procedure for analyzing random walk data (a simplified state-transition process) to find critical meta-states. As can be seen, estimated Markov features are estimated from the random walk data, those states are represented in a feature space, and via the use of a vertex hunting algorithm, anchor states are identified, and those anchor states reveal meta-states. See, Duan et al., “State Aggregation Learning from Markov Transition Data”, NeurIPS 2019, incorporated herein by reference in its entirety.

While not related to healthcare, FIG. 9B is a diagram of the state embedding method applied to the trajectory of a computer game, which can be readily applied here, and is described in more detail below.

High-dimensional time series is ubiquitous in scientific studies and machine learning. Finding compact representation from state-transition trajectories is often a prerequisite for uncovering the underlying physics and making accurate predictions. Suppose that one is given a Markov process {Xₜ} taking values in Ω⊂R^{d}. Let p(y|x) be the one-step transition density function (transition kernel) of the Markov process. In practice, state-transition trajectories may appear high-dimensional, but they are often generated by a system with fewer internal parameters and small intrinsic dimension.

This example focuses on problems where the transition kernel p(y|x) admits a low-rank decomposition structure. Low-rank or nearly low-rank nature of the transition kernel has been widely identified in scientific and engineering applications, e.g. molecular dynamics, periodized diffusion process, traffic transition data, Markov decision process and reinforcement learning. For reversible dynamical systems, leading eigenfunctions of p are related to metastable sets and slow dynamics. Low-rank latent structures also helps state representation learning and dimension reduction in robotics and control.

One goal is to estimate the transition kernel p(⋅|⋅) from finite time series and find state representation in lower dimensions. For nonparametric estimation of probability distributions, one natural approach is the kernel mean embedding (KME). The approach in this example starts with a kernel space, but it “opens up” the kernel function into a set of features. One can leverage the low-rankness of p in the spirit of diffusion map for dimension reduction. By using samples of transition pairs {(Xₜ, Xₜ₊₁)}, one can estimate the “projection” of p onto the product feature space and finds its leading singular functions. This allows one to learn state embeddings that preserve information about the transition dynamics. The approach can be thought of as a generalization of diffusion map to nonreversible processes and Hilbert space. It can be shown that, when the features can fully express the true p, the estimated state embeddings preserve the diffusion distances and can be further used to cluster states that share similar future paths, thereby finding metastable sets and long-term dynamics of the process.

This example illustrates at least four aspects of the process.

KME Reshaping for more accurate estimation of p. The method of KME reshaping is proposed to estimate p from dependent time series data. The method takes advantage of the low-rank structure of p and can be implemented efficiently in compact space. Theorem 1 gives a finite-sample error bound and shows that KME reshaping achieves significantly smaller error than plain KME.

State embedding learning with statistical distortion guarantee. In light of the diffusion map, it studies state embedding by estimating the leading spectrum of the transition kernel. Theorems 2,3 show that the state embedding largely preserves the diffusion distance.

State clustering with misclassification error bound. Based on the state embeddings, states can be further aggregated to preserve the transition dynamics and find metastable sets. Theorem 4 establishes the statistical misclassification guarantee for continuous-state Markov processes.

Experiments with diffusion process and Atari game. The first experiment studies a simulated stochastic diffusion process, where the results validate the theoretical bounds and reveals metastable structures of the process. The second experiment studies the time series generated by a deep Q-network (DQN) trained on an Atari game. The raw time series is read from the last hidden layer as the DQN is run. The state embedding results demonstrate distinctive and interpretable clusters of game states. Remarkably, it is observed that game states that are close in the embedding space share similar future moves, even if their raw data are far different.

### KME Reshaping for Estimating p

In this section the estimation of transition function p from a finite trajectory {Xₜ}ₜ₌₁ⁿ⊂R^{d}. One can make the following low-rank assumption regarding the transition kernel p, which is key to more accurate estimation.

Assumption 1. There exist real-valued functions {uₖ}ₖ₌₁^{r}, {vₖ}ₖ₌₁^{r} on Ω such that p(y|x):=Σₖ₌₁^{r}σₖuₖ(x)vₖ(y), where r is the rank.

Due to the asymmetry of p(⋅,⋅) and lack of reversibility, two reproducing kernel Hilbert spaces H and \tilde{H} can be used to embed the left and right side of p. Let K and \tilde{K} be the kernel functions for H and \tilde{H} respectively. The Kernel Mean Embedding (KME) μₚ(x,y) of the joint distribution p(x,y) into the product space H×\tilde{H} is defined by

μₚ(x,y):=∫K(x,u)\tilde{K}(y,v)p(u,v)dudv.

Given sample transition pairs {Xᵢ,X′ᵢ}ᵢ₌₁ⁿ, the natural empirical KME estimator is

$${\tilde{µ}}_{p} ( x , y ) = \frac{1}{n} {\sum }_{i = 1}^{n} K ( {X}_{i} , x ) \tilde{K} ( {X}_{i}^{′} , y ) .$$

If data pairs are independent, one can show that the embedding error ∥μₚ−\tilde{μ}ₚ∥_{H×\tilde{H}} is approximately

$$\sqrt{\frac{{𝔼}_{( X , Y ) ∼ p} [ K ( X , X ) \tilde{K} ( Y , Y ) ]}{n}} .$$

Next a sharper KME estimator is proposed.

Suppose that the kernel functions K and \tilde{K} are continuous and symmetric semi-definite. Let {Φⱼ(x)}_{j∈J} and {\tilde{Φ}ⱼ(x)}_{j∈J} be the real-valued feature functions on Ω such that K(x,y)=Σ_{j∈J}Φⱼ(x)Φⱼ(y) and \tilde{K}(x,y)=Σ_{j∈J}\tilde{Φ}ⱼ(x)\tilde{Φ}ⱼ(y). In practice, if one is given a shift-invariant symmetric kernel function, one can generate finitely many random Fourier features to approximate the kernel. In what follows it is assumed without loss of generality that J is finite of size N. Let Φ(x)=[Φ₁(x), . . . , Φ_{N}(x)]^{T}∈R^{N}. One can define the “projection” of p onto the feature space by

P=∫p(x,y)Φ(x)\tilde{Φ}(y)^{T}dxdy. (1)

Assumption 1 of this example suggests that rank(P)≤r. Note that the KME of p(x,y) is equivalent to μₚ(x,y)=Φ(x)^{T}P\tilde{Φ}(y). The matrix P is of finite dimensions, therefore one can estimate it tractably from the trajectory {Xₜ} by

$$\hat{P} := \frac{1}{n} {\sum }_{t = 1}^{n} Φ ( {X}_{t} ) {\tilde{Φ} ( {X}_{t + 1} )}^{T} . \qquad ( 2 )$$

Since the unknown P is low-rank, we propose to apply singular value truncation to \tilde{P} for obtaining a better KME estimator. The algorithm is given below:

Algorithm 1: Reshaping the Kernel Mean Embedding.

Input: {X₁, . . . , Xₙ}, r;
Get \hat{P} by (2), compute its SVD: \hat{P} = Û\hat{Σ}\hat{V};
Let \tilde{P} := Û\hat{Σ}_{[1. . . r]} \hat{V} be the best rank r approximation of \hat{P};
Let \hat{μ}ₚ (x, y) := Φ (x)^{T}\tilde{P}\tilde{Φ}(y);
Output: \hat{μ}ₚ (x, y)

One can analyze the convergence rate of \tilde{μ}ₚ to μₚ. Let Kₘₐₓ:=max{sup_{x∈Ω}K(x,x), sup_{x∈Ω}\tilde{K}(x,x)}. One can define the following kernel covariance matrices:

V₁= [symbol] _{(X,Y)˜p}[Φ(X)Φ(X)^{T}\tilde{K}(Y,Y)], V₂= [symbol] _{(X,Y)˜p}[K(X,X)\tilde{Φ}(Y)\tilde{Φ}(Y)^{T}].

Let λ̅:=max {λₘₐₓ(V₁),λₘₐₓ(V₂)}. The following finite-sample error bound can be shown.

Theorem 1 (KME Reshaping). Let Assumption 1 hold. For any δ∈(0,1), we have

$${‖ {μ}_{p} - {\hat{μ}}_{p} ‖}_{ℋ \times \ddot{ℋ}} = {‖ P - \tilde{P} ‖}_{F} \leq C \sqrt{r} ( \sqrt{\frac{{t}_{mix} \overline{λ} log ( 2 {t}_{mix} N / δ )}{n}} + \frac{{t}_{mix} {K}_{max} log ( 2 {t}_{mix} N / δ )}{3 n} )$$

with probability at least 1−δ, where C is a universal constant.

The KME reshaping method and Theorem 1 enjoys the following advantages:

1. Improved accuracy compared to plain KME. The plain KME \tilde{μ}ₚ's estimation error is approximately

$$\sqrt{\frac{{E}_{( X , Y ) ∼ p} [ K ( X , X ) \tilde{K} ( Y , Y ) ]}{n}} .$$

Note that Tr(V₁)=Tr(V2)=E_{(X,Y)˜p}[K(X,X)\tilde{K}(Y,Y)]. When r<<N, one typically has rλ̅<<Tr(V₁)=Tr(V₂), therefore the reshaped KME has a significantly smaller estimation error.

2. Ability to handle dependent data. Algorithm 1 applies to time series consisting of highly dependent data. The proof of Theorem 1 handles dependency by constructing a special matrix martingale and using the mixing properties of the Markov process to analyze its concentration.

3. Tractable implementation. Kernel-based methods usually require memorizing all the data and may be intractable in practice. The current approach is based on a finite number of features and only needs to low-dimensional computation. One can approximate any shift-invariant kernel function using N features where N is linear with respect to the input dimension d. Therefore Algorithm 1 can be approximately implemented in O(nd²) time and O(d²) space.

### Embedding States into Euclidean Space

In this section, low-dimensional representations of the state space Ω to capture the transition dynamics are learned. One needs the following extra assumption that p can be fully represented in the kernel space.

Assumption 2. The transition kernel belongs to the product Hilbert space, i.e., p(⋅|⋅)∈H×\tilde{H}.

For two arbitrary states x, y∈Ω, one can consider their distance given by

dist(x,y):=∥p(⋅|x)−p(⋅|y)∥_{L}_{²}=(∫(p(z|x)−p(z|y))²dz)^{1/2}. (3)

Eq. (3) is known as the diffusion distance. It measures the similarity between future paths of two states. One is motivated by the diffusion map approach for dimension reduction. Diffusion map refers to the leading eigenfunctions of the transfer operator of a reversible dynamical system. One can generalize it to nonreversible processes and feature spaces.

For simplicity of presentation, we assume without loss of generality that {(Φᵢ}ᵢ₌₁^{N} and {\tilde{Φ}ᵢ}ᵢ₌₁^{N} are L₂(Π) and L₂ orthogonal bases of H and \tilde{H} respectively, with squared norms ρ₁≥ . . . ≥ρ_{N} and \tilde{ρ}₁≥ . . . ≥\tilde{ρ}_{N} respectively. Any given features can be orthogonalized to satisfy this condition. In particular, let the matrix C:=ρ₁, . . . , ρ_{N}, \tilde{C}:=\tilde{ρ}₁, . . . , \tilde{ρ}_{N}, it is easy to verify that p(y|x)=Φ(x)^{T}C⁻¹P\tilde{C}⁻¹\tilde{Φ}(y). Let C^{-1/2}P\tilde{C}^{-1/2}=U^{(ρ)}Σ_{[1 . . . r]}^{(ρ)}V^{(ρ)} be its SVD. One can define the state embedding as

Ψ(x):=(Φ(x)^{T}C^{-1/2}U^{(ρ)}Σ_{[1 . . . r]}^{(ρ)})^{T}.

It is straightforward to verify that dist(x,z)=∥Ψ(x)−Ψ(z)∥. Algorithm 2 can be used to estimate Ψ.

Algorithm 2: Learning State Embedding

Input: {X₁, . . . , Xₙ}, r;
Get \hat{P} from (2), compute SVD Û^{(ρ)}\hat{Σ}^{(ρ)}\hat{V}^{(ρ)} = C^{-1/2}\hat{P}\tilde{C}^{-1/2};
Compute state embedding using first r singular pairs \hat{Ψ} (x) = (Φ (x)^{T}C^{-1/2}Û^{(ρ)}\hat{Σ}_{[1 . . . r]}^{(ρ)})^{T};
Output: x [symbol] \hat{Ψ} (x)

Let [symbol] (x,z):=∥\hat{Ψ}(x)−\hat{Ψ}(z)∥. One can show that the estimated state embeddings preserve the diffusion distance with an additive distortion.

Theorem 2 (Maximum additive distortion of state embeddings). Let Assumptions 1,2 hold. Let Lₘₐₓ:=sup_{x∈Ω}Φ(x) and let κ be the condition number of \sqrt{π(x)}p(y|x). For any 0<δ<1 and for all x, z∈Ω, |dist(x,z)− [symbol] (x,z)| is upper bounded by:

$$C \sqrt{\frac{{L}_{ma x}}{{ρ}_{N} {\tilde{ρ}}_{N}}} [ \sqrt{2} κ + 1 ] ( \sqrt{\frac{{t}_{mix} \overline{λ} log ( 2 {t}_{mix} N / δ )}{n}} + \frac{{t}_{mix} {K}_{ma x} log ( 2 {t}_{mix} N / δ )}{3 n} )$$

with probability at least 1−δ for some constant C.

Under Assumption 2, we can recover the full transition kernel from data by

\hat{p}(y|x)=Φ(x)^{T}C^{-1/2}Û^{(ρ)}\hat{Σ}_{[1 . . . r]}^{(ρ)}(\hat{V}^{(ρ)})^{T}\tilde{C}^{-1/2}\tilde{Φ}(y).

Theorem 3 (Recovering the transition density). Let Assumptions 1,2 hold. For any δ∈(0, 1),

$${‖ p ( \cdot \vert \cdot ) - \hat{p} ( \cdot \vert \cdot ) ‖}_{{L}^{2} ( π ) \times {L}^{2}} \leq C \sqrt{\frac{r}{{ρ}_{N} {\tilde{ρ}}_{N}}} ( \sqrt{\frac{{t}_{mix} \overline{λ} log ( 2 {t}_{mix} N / δ )}{n}} + \frac{{t}_{mix} {K}_{ma x} log ( 2 {t}_{mix} N / δ )}{3 n} )$$

with probability at least 1−δ for some constant C.

Theorems 2,3 provide the first statistical guarantee for learning state embeddings and recovering the transition density for continuous-state low-rank Markov processes. The state embedding learned by Algorithm 2 can be represented in O(Nr) space since Φ is priorly known. When Ω is finite and the feature map is identity, Theorem 3 nearly matches the information-theoretical error lower bound. See, Li et al, “Estimation of Markov chain via rank-constrained likelihood”, International Conference on Machine Learning, 2018, incorporated herein by reference in its entirety.

### Clustering States Using Diffusion Distances

A partition of the state space into m disjoint sets Ω₁ . . . Ωₘ can be found. The principle is if x, y∈Ωᵢ for some i, then p(⋅|x)≈p(⋅|y), meaning that states within the same set share similar future paths. The following optimization problem can be studied, which has previously been considered in studies for dynamical systems,

$${min}_{{Ω}_{1} , … , {Ω}_{m}} {min}_{{q}_{1} ∈ \ddot{ℋ} , … , {q}_{m} ∈ \ddot{ℋ}} {\sum }_{i = 1}^{m} {\int }_{{Ω}_{i}} π ( x ) {‖ p ( \cdot \vert x ) - {q}_{i} ( \cdot ) ‖}_{{L}^{2}}^{2} dx , \qquad ( 4 )$$

It is assumed without loss of generality that it admits a unique optimal solution, which is denoted by (Ω₁*, . . . , Ωₘ*) and (q₁*, . . . , qₘ*). Under Assumption 2, each qᵢ*(⋅) is a probability distribution and can be represented by right singular functions {vₖ(⋅)}ₖ₌₁^{r} of p(⋅|⋅). The following state clustering method is proposed:

Algorithm 3: Learning metastable state clusters

Data: {X₁, . . . , Xₙ, r, m}
Use Alg. 2 to get state embedding \hat{Ψ} : Ω [symbol] [symbol] ^{r};
Solve k-means problem:
${min}_{{Ω}_{1} , … , {Ω}_{m}} {{\sum }^{m}}_{i = 1}^{ } {\int }_{{Ω}_{i}}^{ } π ( x ) {‖ \hat{Ψ} ( x ) - {s}_{i} ‖}^{2} dx ;$
Output: \hat{Ω}₁^{*}, . . . , \hat{Ω}ₘ^{*}

The k-means method uses the invariant measure Π as a weight function. In practice if Π is unknown, one can pick any reasonable measure and the theoretical bound can be adapted to that measure.

One can analyze the performance of the state clustering method on finite data. Define the misclassification rate as

$$M ( {\hat{Ω}}_{1}^{*} , … , {\hat{Ω}}_{m}^{*} ) := {min}_{σ} {\sum }_{j = 1}^{m} \frac{π ( \{ x \text{:} x ∈ {Ω}_{j}^{*} , i ∉ {\hat{Ω}}_{σ ( j )}^{*} \} )}{π ( {Ω}_{j}^{*} )} ,$$

where σ is taken over all possible permutations over {1, . . . , m}. The misclassification rate is always between 0 and m. One can let Δ₁²:=minₖmin_{l≠k}π(Ωₖ*)∥qₗ*−qₖ*∥_{L}_{²}² and let Δ₂² be the minimal value of (4).

Theorem 4 (Misclassification error bound for state clustering). Let Assumptions 1,2 hold. Let κ be the condition number of \sqrt{π(x)}p(y|x). If Δ₁>4Δ₂, then for any 0<δ<1 and ∈>0, by letting

$$n = Θ ( \frac{{κ}^{2} r \overline{λ } {t}_{m i x} log ( 2 {t}_{mix} N / δ )}{{ρ}_{N} {\tilde{ρ}}_{N}} \cdot max \{ \frac{1}{{( {Δ}_{1} - 4 {Δ}_{2} )}^{2}} , \frac{1}{ϵ {Δ}_{1}^{2}} , \frac{{Δ}_{2}^{2}}{{ϵ}^{2} {Δ}_{1}^{4}} \} ) , \\ one has M ( {\hat{Ω}}_{1}^{*} , … , {\hat{Ω}}_{m}^{*} ) \leq \frac{16 {Δ}_{2}^{2}}{{Δ}_{1}^{2}} + ϵ with probability at 1 - δ .$$

The condition Δ₁>4Δ₂ is a separability condition needed for finding the correct clusters with high probability, and

$$\frac{16 {Δ}_{2}^{2}}{{Δ}_{1}^{2}}$$

is non-vanishing misclassification error. In the case of reversible finite-state Markov process, the clustering problem is equivalent to finding the optimal metastable m-full partition given by argmax_{Ω}_{₁}_{, . . . ,Ω}_{ₘ} Σₖ₌₁^{m}p(Ωₖ|Ωₖ), where

$$P ( {Ω}_{j} \vert {Ω}_{i} ) : = \frac{1}{π {Ω}_{i}} {\int }_{x ∈ {Ω}_{i} , y ∈ {Ω}_{j}} π ( x ) p ( y \vert x ) d y d x .$$

The optimal partition (Ω₁, . . . , Ωₘ) gives metastable sets that can be used to construct a reduced-order Markov state model. In the more general case of nonreversible Markov chains, the proposed method will cluster states together if they share similar future paths. It provides an unsupervised learning method for state aggregation, which is a widely used heuristic for dimension reduction of control and reinforcement learning.

### Experiments

### Stochastic Diffusion Processes

One can test the proposed approach on simulated diffusion processes of the form dXₜ=−∇V(Xₜ)dt+\sqrt{2}dBₜ, Xₜ∈R^{d}, where V(⋅) is a potential function and {Bₜ}_{t≥0} is the standard Brownian motion. For any interval τ>0, the discrete-time trajectory {X_{kτ}}ₖ₌₁^{∞} is a Markov process. One can apply the Euler method to generate sample path {Xₖᵣ}ₖ₌₁ⁿ according to the stochastic differential equation.

One can use the Gaussian kernels

$$K ( x , y ) = \tilde{K} ( x , y ) = \frac{1}{{( 2 π {σ}^{2} )}^{d / 2}} {e}^{- \frac{{‖ x - y ‖}_{2}^{2}}{2 {σ}^{2}}}$$

where σ>0, and construct RKHS H=\tilde{H} from L²(π). To get the features Φ, 2000 random Fourier features h=[h₁, h₂, . . . , h_{N}]^{τ} are generated such that K(x,y)≈Σᵢ₌₁^{N}hᵢ(x)hᵢ(y), and then orthogonalize h to get Φ.

### Comparison Between Reshaped and Plain KME

One can apply Algorithm 1 to find the reshaped KME \hat{μ}ₚ and compare its error with the plain KME \hat{μ}ₚ given by (2). The experiment is performed on a four-well diffusion on R, and rank r=4 was taken. By orthogonalizing N=2000 random Fourier features, J=82 basis functions were obtained. The reshaped KME consistently outperforms plain KME with varying sample sizes.

### State Clustering to Reveal Metastable Structures

One can apply the state clustering method to analyze metastable structures of a diffusion process with a potential function V(x). One can generate trajectories of length n=10⁶ and take the time interval to be τ=0.1, 1,5 and 10. One can conduct the state embedding and clustering procedures with rank r=4. The partition results reliably reveal metastable sets, which are also known as invariance sets that characterize slow dynamics of this process. The contours are based on diffusion distances to the centroid in each cluster. One can see that the diffusion distance contours are dense when τ takes small values. This is because, when τ is small, the state embedding method largely captures fast local dynamics. By taking τ to be larger values, the state embedding method begins to capture slower dynamics, which corresponds to low-frequency transitions among the leading metastable sets.

### DQN for Demon Attack

In this example, the state embedding method was tested on the game trajectories of Demon Attack, an Atari 2600 game. In this game, demons appear in waves, move randomly and attack from above, where the player moves to dodge the bullets and shoots with a laser cannon. A Deep Q-Network (DQN) was trained using the architecture given by Mnih et al, “Human-level control through deep reinforcement learning”, Nature, 518:529-533, 2015.

As roughly depicted in FIG. 9B, the DQN takes recent image frames of the game as input, and processes them through three convolutional layers and two fully connected layers, and outputs a single value for each action, among which the action with the maximal value is chosen.

In this environment, each game frame oₜ is a 210×160×3 image. In each interactive step the agent takes in the last 16 frames and preprocesses them to be the input state sₜ=ϕ({oₜ₋ᵢ}ᵢ₌₀¹⁵). The state sₜ is an 84×84×4 resealed, grey-scale image, and is the input to the neural network Q (sₜ,⋅;θ). The first convolution layer in the network has 32 filters of size 8 stride 4, the second layer has 64 layers of size 4 stride 2, the final convolution layer has 64 filters of size 3 stride 1, and is followed by a fully-connected hidden layer of 512 units. The output is another fully-connected layer with six units that correspond to the six action values {Q(sₜ, aᵢ;θ)}ᵢ₌₁⁶. The agent selects an action based on these state-action values, repeats the selected action four times, observes four subsequent frames {oₜ₊ᵢ}ᵢ₌₁⁴ and receives an accumulated reward rₜ.

The agent in the DQN algorithm “learns” through a novel variant of Q-learning that employs the techniques of target net (θ−) and experience replay (D), and conducts gradient descent to the following loss function at each iteration:

$$ℒ ( θ ) = {E}_{s , a , r , {s}^{′} \sim U ( D )} [ {( r + {γmax}_{{a}^{′}} {Q ( {s}^{′} , {a}^{′} ; {θ}^{-} )}^{2} - Q ( s , a ; θ ) )}^{2} ] .$$

Unlike Minh, this method uses an Adam optimizer (see Kingma et al, “Adam: A method for stochastic optimization”, In 3rd International Conference on Learning Representations, ICLR 2015, San Diego, Calif., USA, May 7-9, 2015, Conference Track Proceedings, 2015, incorporated herein by reference in its entirety), with a decaying learning rate, and a smaller replay buffer of size 500 k frames. Training is done over 2.5 million steps, i.e., 10 million game frames. The Q-network is stored and evaluated every 500 k steps. The best policy among these evaluations attains a 150-200% human-level performance, and is later used as the sampling policy.

The raw input to the state embedding algorithm is a time series of length 47936 and dimension 512, comprising 130 trajectories generated by the fully connected hidden layer in DQN when it is running the sampling policy. The embeddings are obtained through the same process as in Experiment 6.1 with a Gaussian kernel and 200 random Fourier features. The rank r is set to be 3, and the time interval τ corresponds to 12 game frames, i.e., 0.36 second in real time. Both the raw date and the embeddings are projected onto 2D planes by t-SNE with a perplexity of 40.

In this experiment, the times series generated by the last hidden layer of a trained DQN when playing the game is taken as the raw data. The raw data is a time series of length 47936 and dimension 512, comprising 130 game trajectories. One can apply the state embedding method by approximating the Gaussian kernel with 200 random Fourier features. Then one can obtain low-dimensional embeddings of the states in R³.

### Before Embedding vs. After Embedding

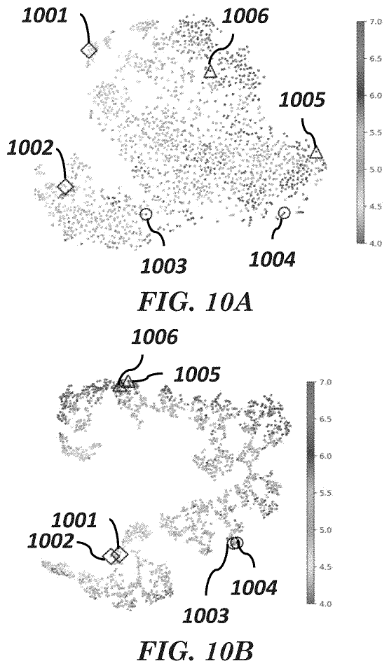

FIGS. 10A and 10B visualize the raw states and the state embeddings using t-SNE, a visualization tool to illustrate multi-dimensional data in two dimensions. In both plots (before—FIG. 10A—and after—FIG. 10B—embedding), states that are mapped to nearby points tend to have similar “values” (expected future returns) as predicted by the DQN, as illustrated by colors of data points. Comparing the two plots, the raw state data are more scattered, while after embedding they exhibit clearer structures and fewer outliers. The markers (diamonds, 1001, 1002; circles 1003, 1004; triangles 1005, 1006) used to identify the same pair of game states before and after embedding suggest that the embedding method maps game states that are far apart from each other in their raw data to closer neighbors. It can be viewed as a form of compression. The experiment has been repeated multiple times. It is consistently observed that state embedding leads to improved clusters and higher granularity in the t-SNE visualization.

### Example 3

This example describes an alternative to the previous state embedding method, utilizing dimension reduction of both states and actions.

State abstraction is a core problem at the heart of control and reinforcement learning (RL). In high-dimension RL, a naive grid discretization of the continuous state space often leads to exponentially many discrete states—an open challenge known as the curse of dimensionality. Having good state representations will significantly improve the efficiency of RL, by enabling the use of function approximation to better generalize knowledge from seen states to unseen states.

One can say a state/action representation is “good”, if it enables the use of function approximation to extrapolate and predict future value of unseen states. Suppose there is a representation allowing exact linear parametrization of the transition and value functions, then the sample complexity of RL reduces to depend linearly on d—the representation's dimension. Even if exact parametrization is not possible, a good representation can be still useful for solving RL with approximation error guarantee. An important related problem is to strategically explore in online RL while learning state abstractions. One desires methods that can learn good representations, for RL with high-dimensional state and action spaces, automatically from empirical data.

What further complicates the problem is the large action space. An action can be either a one-step decision or a sequence of multi-step decisions (known as option). States under different actions lead to very different dynamics. Although states and actions may admit separate low-dimensional structures, they are entangled with each other in sample trajectories. This necessitates the tensor approach to decouple actions from states, so that one can learn their abstractions simultaneously and respectively.

The state and action abstraction of Markov decision processes (MDP) can be studied from a tensor decomposition view. It may be useful to focus on the batch data setting. The Tucker decomposition structure of a transition kernel p provides natural abstractions of the state and action spaces. The low-Tucker-rank property in a number of reduced-order MDP models is illustrated, including the block MDP (aka hard aggregation), latent-state model (aka soft aggregation).

Suppose one is given state-action-state transition samples D={(s, a, s′)} from a long sample path generated by a behavior policy. One objective in this example is to identify a state embedding map and an action embedding map, which map the original state and action spaces (maybe continuous and high-dimensional) into low-dimensional representations, respectively. The embedding maps are desired to be maximally “predicative”, by preserving a notion of kernelized diffusion distance that measures similarity between states in terms of their future dynamics.

To handle continuous state and action spaces, one can use non-parametric function approximation with known kernel functions over the state and action spaces. By approximately decomposing the kernel into finitely many features, one is able to handle the continuous problem by estimating a transition tensor of finite dimensions. Next, importance sampling and low-rank tensor approximation are leveraged to identify the desired state and action embedding maps. They yield “good” representations of states and actions that are useful for linear function approximation in RL. Further, these representations can be used to find the best discrete approximation to the MDP, and in particular, recover the latent structures of block MDP with high accuracy.

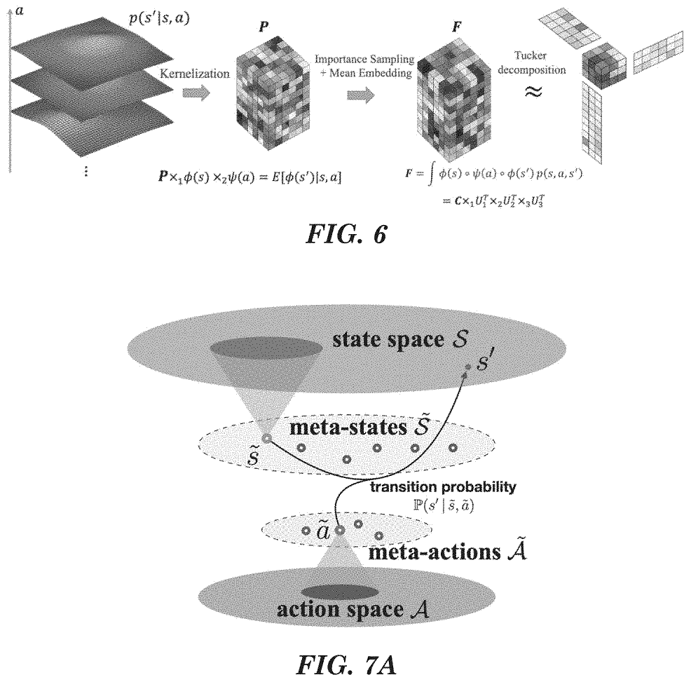

FIG. 6 illustrates the main idea of this approach, which includes:

(i) a tensor-inspired kernelized embedding method to learn low-dimensional state and action representations from empirical trajectories. The method exploits the MDP's tensor structure by importance sampling, mean embedding and low-rank approximation.

(ii) Theoretical guarantee that the embedding maps largely preserve the “predictability” of states and actions in terms of a kernelized diffusion distance, which is proved using a novel tensor concentration analysis.

(iii) A discrete state/action abstraction method that provably recovers latent block structures of aggregable MDP.

(iv) Theoretical guarantee that the learned abstractions are “good” representations for approximating transition/value functions within a small error tolerance.

Additionally, the tensor-inspired approach is computation- and memory-efficient, via the use of random features and tensor power iteration.

### Markov Decision Process

An instance of a Markov decision process can be specified by a tuple M=(S,A,p,r,ρ), where S and A are state and action spaces, p is the transition probability kernel, r:S×A→R is the one-step reward function, ρ is the initial state distribution. At each step t, suppose the current state is sₜ. If the agent chooses an action aₜ, she will receive an instant reward r(sₜ, aₜ)∈[0, 1] and system's state will transit to sₜ₊₁ according to the probability distribution p(⋅|sₜ, aₜ).

A policy Π is a rule for choosing actions based on states, where Π(⋅|s) is a probability distribution over A conditioned on s∈S. Under a given policy, the transition of the MDP will reduce to a Markov chain, whose transition kernel is denoted by p^{Π} where p^{Π} (s′|s)=p^{1,Π}(s′|s)=∫_{A} π(a|s)p(s′|s,a)da. Based on that, one can define the t-step transition kernel p^{t,Π}(⋅|s) inductively by p^{t,Π}(⋅|s)=∫p^{t-1,π}(s′|s)p^{π}(⋅|s′)ds′. And one can further use v^{Π to denote the invariant distribution of that Markov chain. Define the worst-case mixing time as}

$${t}_{mix} = {max}_{π} min \{ t \vert {‖ {p}^{\tilde{t} , π} ( \cdot \vert {s}_{0} ) - {v}^{π} ‖}_{TV} \leq \frac{1}{4} , ∀ {s}_{0} ∈ 𝒮 , \tilde{t} \geq t \} ,$$

where ∥⋅∥_{TV} denotes the total variation distance. This example focuses on fast-mixing MDP such that tₘᵢₓ<<∞.

Tensor and Tucker Decomposition.

For a general tensor X∈R^{p}^{₁}^{×p}^{₁}^{× . . . p}^{_{N}}, we denote X×n U as the product between X and a matrix U∈R^{p}^{ₙ}^{×q} on the n^{th} dimension, which is of size p₁× . . . ×pₙ₋₁×q×pₙ₊₁× . . . ×p_{N}. Each element of X×ₙ U is defined as

$${( X {\times }_{n} U )}_{{i}_{1} … {i}_{n - 1} {ji}_{n + 1} … {i}_{N} } = {\sum }_{i = 1}^{{p}_{n}} {X}_{{i}_{1} … {i}_{n - 1} {ii}_{n + 1} … {i}_{N}} {U}_{ij} .$$

The factor-k matricization (or flattening) of X is denoted by Mₖ(X)∈R^{p}^{ₖ}^{×Π}^{_{i≠k}}^{p}^{ᵢ}. The Tucker decomposition of X is of the form X=G×₁U₁×_{2 . . .} ×_{N} U_{N}, where G∈R^{q}^{₁}^{×q}^{₁}^{× . . . q}^{_{N}}, is a smaller core tensor. In particular, one can call the smallest size of G the Tucker-rank of X. Rigorously, one can define Tucker-Rank(X)=(R₁, R₂, . . . , R_{N}), where Rₖ=Rank(Mₖ(X)). The inner product between two tensors X, Y∈R^{p}^{₁}^{×p}^{₂}^{× . . . p}^{_{N}} is defined as

$$〈 X , Y 〉 = {\sum }_{{i}_{1} = 1}^{{p}_{1}} {\sum }_{{i}_{2} = 1}^{{p}_{2}} … {\sum }_{{i}_{N} = 1}^{{p}_{N}} {X}_{{i}_{1} {i}_{2} … {i}_{N}} {Y}_{{i}_{1} {i}_{2} … {i}_{N}}$$

The spectral norm and Frobenius norm of a tensor X∈R^{p}^{₁}^{×p}^{₂}^{× . . . p}^{_{N}} are defined as

$${‖ X ‖}_{σ} = {sup}_{‖ {u}_{i} ‖ = 1 , 1 \leq i \leq N} 〈 X , {u}_{1} ∘ {u}_{2} ∘ … ∘ {u}_{N} 〉$$

$${‖ X ‖}_{F} = \sqrt{〈 X , X 〉} = \sqrt{{\sum }_{{i}_{1} = 1}^{{p}_{1}} … {\sum }_{{i}_{N} = 1}^{{p}_{N}} {X}_{{i}_{1} {i}_{2} … {i}_{N}}^{2}} .$$

A Tensor View of Markov Decision Process

Consider a continuous-state MDP with the transition kernel p, where each p(⋅|s, a) is a conditional transition density function. One can adopt a tensor view to exploit structures of p for abstractions of state and action spaces. The Tucker rank of p turns out related to commonly used reduced-order models such as state aggregation and latent models.

One can handle the continuous state and action spaces using kernel function approximation. Suppose one has a Reproducing Kernel Hilbert Space (RKHS) H_{S} for functions over states and a RKHS H_{A} for functions over actions. We make the assumption that the MDP's transition kernel p can be represented in these function spaces.

Assumption 1. Let [symbol] be the transition operator of p, i.e., ( [symbol] ƒ)(s,a)=∫p(s′|s,a)ƒ(s′)ds′. Assume that [symbol] ƒ∈H_{S}×H_{A}, ∀ƒ∈H_{S} and Tucker-Rank( [symbol] )≤(r, l, m).

Here, the Tucker-rank of an operator is defined as follows: there exists cᵢⱼₖ∈R and functions uᵢ,wₖ∈H_{S},vⱼ∈H_{A},i∈[r],j∈[l],k∈[m], such that

$$( ℙ f ) ( s , a ) = {\sum }_{i = 1}^{r} {\sum }_{j = 1}^{l} {\sum }_{k = 1}^{m} {c}_{ijk} {u}_{i} ( s ) {v}_{j} ( a ) {〈 f , {w}_{k} 〉}_{{ℋ}_{S}}$$

In this example, it is assumed without loss of generality that the state kernel space and action kernel space admit finitely many known basis functions, which are referred to as state features ϕ(s)∈R^{d}^{ₛ} and action features ψ(a)∈R^{d}^{_{A}}.

This is a rather mild assumption: Even if one does not know the basis function but are only given kernel functions K₁ and K₂ for H_{S} and H_{A}. One can always generate finitely many random features to approximately span these kernel spaces such that K₁(s,s′)≈Σᵢ₌₁^{d}^{ₛ}ϕᵢ(s)^{τ}ϕᵢ(s′) and K₂(a,a′)≈Σᵢ₌₁^{d}^{_{A}}ψᵢ(a)^{τ}ψᵢ(a′). Also note that this approach applies to arbitrary state and action spaces, as long as they come with appropriate kernel functions.

Although p is infinitely dimensional, one can use the given kernel spaces and represent p with a finite-dimensional tensor. In particular, Assumption 1 implies the following tensor linear model:

Lemma 1 (Conditional transition tensor and linear model). Suppose Assumption 1 holds. There exists a tensor P∈R^{d}^{_{S}}^{×d}^{_{A}}^{×d}^{_{S}} such that

P×₁ϕ(s)×₂ψ(a)= [symbol] [ϕ(s′)|s,a], ∀s,a

### and Tucker-Rank(P)≤(r, l, m).

Tucker decomposition is one of the most general low-rank structure for tensors. A stronger notion of tensor decomposition is the CP decomposition. Note that a CP-rank-r tensor is naturally Tucker rank-(r, r, r) but not vice versa.

Remarkably, the low-Tucker-rank property (Assumption 1) turns out to be universal in a number of reduced-order MDP models. Here are two basic examples.

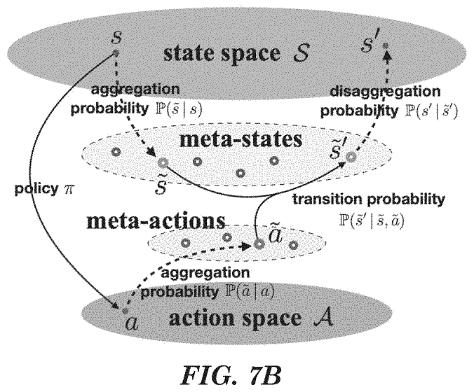

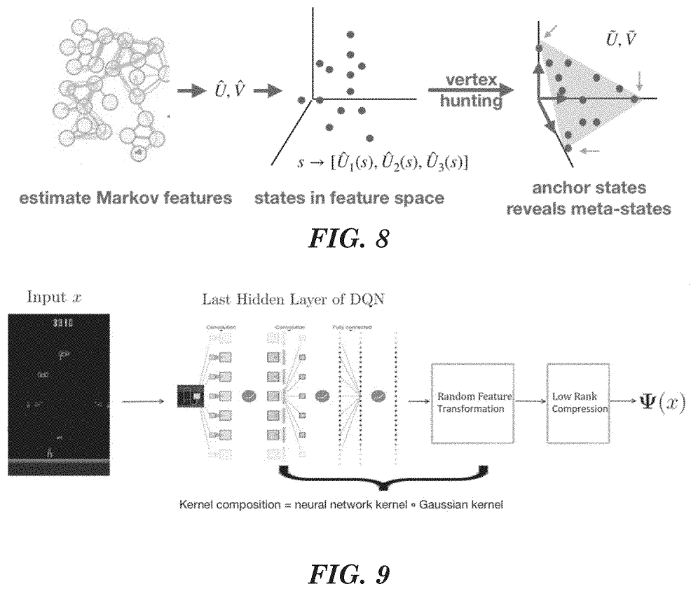

Example A (See FIG. 7A, Block MDP (Hard Aggregation). Let \tilde{S} and Ã be finite sets. Suppose there exists state and action abstraction ƒ:S [symbol] \tilde{S} and g:S [symbol] Ã such that

p(⋅|s,a)=p(⋅|s′,a′) if ƒ(s)=ƒ(s′),g(a)=g(a′)

Then p has Tucker rank at most (|\tilde{S}|, |Ã|, |S|).

Example B (See FIG. 7B, Latent-State-Action MDP (Soft Aggregation)). Given an MDP M=(S,A,p,r) we say M has an (R, l, m)-latent variable model if there exist a latent state-action-state stochastic process {\tilde{s}ₜ, ãₜ, \tilde{s}′ₜ}⊆\tilde{S}×Ã×\tilde{S}′, with |\tilde{S}|=r, |Ã|=l, |\tilde{S}′|=m, such that

[symbol]

(\tilde{s}ₜ,ãₜ|s₁,a₁, . . . ,sₜ,aₜ)= [symbol] (\tilde{s}ₜ|sₜ) [symbol] (ãₜ|aₜ),

[symbol]

(\tilde{s}′ₜ|s₁,a₁, . . . ,sₜ,aₜ,\tilde{s}ₜ,ãₜ)= [symbol] (\tilde{s}′ₜ|\tilde{s}ₜ,ãₜ),

[symbol]

(sₜ₊₁|s₁,a₁, . . . ,sₜ,aₜ,\tilde{s}ₜ,ãₜ,\tilde{s}′ₜ)= [symbol] (sₜ₊₁|\tilde{s}′ₜ).

In this case, one can verify that p has Tucker rank (r, l, m).

The low-Tucker-rank property also holds in MDPs with rich observations and is related to the Bellman rank. The tensor rank is determined solely by the transition model p (i.e., the environment), regardless of reward r.

Tensor-Inspired State and Action Embedding Learning

A tensor-inspired representation learning method can be developed, that embeds states and actions into decoupled low-dimensional spaces. the method will be developed step by step.

Tensor MDP Mean Embedding by Importance Sampling

Suppose that one has a batch dateset of state-action samples.

Assumption 2. The data D={(s, a, s′)} consists of state-action-state transitions from a single sample path generated by a known behavior policy π̅.

Let ξ be the stationary state distribution of the sample path under policy π̅. This ξ is not known a priori. Let η be a positive probability measure over the action space. Consider the tensor mean embedding

F=∫ϕ(s)oψ(a)oϕ(s′)p(s,a,s′)dsdads′∈ [symbol] ^{d}^{_{S}}^{×d}^{_{A}}^{×d}^{_{S}}

where p(s, a, s)=p(s|s, a)ξ(s)η(a).

Lemma 2. Assumption 1 implies Tucker-Rank(F)>(r, l, m).

One can estimate the mean embedding tensor F by importance sampling:

$$\overline{F} = \frac{1}{n} {\sum }_{i = 1}^{n} \frac{η ( {a}_{i} )}{\ddot{π} ( {a}_{i} \vert {s}_{i} )} φ ( {s}_{i} ) ∘ ψ ( {a}_{i} ) ∘ φ ( {s}_{i}^{′} ) . \qquad ( 1 )$$

It can be easily verified that the estimator is asymptotically “unbiased” by the following lemma.

Lemma 3. If tₘᵢₓ<∞, lim_{n→∞}∥ [symbol] [F̅]−F∥_{F}=0

Furthermore, the mean embedding tensor F is related to the conditional transition tensor P through a simple relation.

Lemma 4 (Relation between P and F). When {ψi(⋅)}ᵢ₌₁^{d}^{_{A}} forms a set of orthogonal basis with respect to L²(η), one has P=F×₁Σ⁻¹, where Σ=ξ(s)ϕ(s)ϕ(s)^{τ}ds.

Necessity of importance sampling. The importance sampling step (1) is necessary to decouple states from actions. Without importance sampling, the naive mean embedding tensor

W:=∫ϕ(s)oψ(a)oϕ(s′)ξ(s)π̅(a|s)p(s′|a,s)dsdads′

may have large ranks on the first two dimensions. This is due to that the behavior policy Π⁻couples the state and action spaces together, therefore their independent low-dimensional structures are lost in the mean embedding tensor W. Without using importance sampling, if we replace F with plain mean W, Lemma 2 and Lemma 4 no longer hold. As a result, one cannot learn the best low-dimensional structure of p from W.

Low-Rank Estimation of Transition Tensor P We estimate a low-rank approximation to F by solving:

\hat{F}=argmin ∥Q−F̅∥_{σ} subject to Tucker-Rank(Q)≤(r,l,m) (2)

Define

$${K}_{ma x} = max \{ {sup}_{s} {K}_{1} ( s , s ) , {sup}_{a} {K}_{2} ( a , a ) \} , \\ \overline{λ} = {sup}_{u , v , w} {𝔼}_{ξ ∘ η ∘ p ( \cdot \vert \cdot )} [ {[ ( {u}^{T} φ ( S ) ) ( {v}^{T} ψ ( A ) ) ( {w}^{T} φ ( {S}^{′} ) ) ]}^{2} ] , \\ where u , w , ∈ {S}^{{d}_{S} - 1} , v ∈ {S}^{{d}_{A} - 1} , \\ \overline{μ} = {‖ 𝔼 [ {K}_{1} ( S , S ) φ ( S ) {φ ( S )}^{T} ] ‖}_{σ} , κ = {sup}_{s ∈ S , a ∈ A} \frac{η ( a )}{π ( a \vert s )} .$$

Theorem 1 (Concentration in tensor spectral norm). Let Assumptions 1-2 hold. Suppose

$$\frac{n / {t}_{mix}}{{( log ( n / {t}_{mix} ) )}^{2}} \geq 1024 \frac{κ {K}_{ma x}^{3}}{\overline{λ}} ( log \frac{{t}_{mix}}{δ} + 8 ( {d}_{S} + {d}_{A} ) ) ,$$

then with probability 1−δ, one has

$${‖ \hat{F} - F ‖}_{σ} \leq 64 \sqrt{\frac{κ \overline{λ} ( log \frac{{t}_{mix}}{δ} + 8 ( {d}_{S} + {d}_{A} ) ) {( log \frac{n}{{t}_{mix}} )}^{2}}{n / {t}_{mix}}} .$$

Computation by tensor power iteration. Since finding the exact optimum of (2) can be computationally intense in general, in practice, applying the higher-order orthogonal iteration (HOOI), a classic computationally efficient algorithm, is proposed to find an approximate solution to (2).

Recall the relation P=F×₁ Σ⁻¹, one can estimate the transition operator P by

$$\hat{P} = \hat{F} {\times }_{1} {\hat{Σ}}^{- 1} , where \hat{\sum } = \frac{1}{n} {\sum }_{i = 1}^{n} φ ( {s}_{i} ) {φ}^{T} ( {s}_{i} ) .$$

Theorem 2 (Low-rank estimation of the transition tensor P). Let Assumptions 1-2 hold. Suppose ψ is orthogonal with respect to L²(η), and

$$\frac{n / {t}_{mix}}{{( log ( n / {t}_{mix} ) )}^{2}} \geq 1024 ( {‖ {Σ}^{- 1} ‖}_{σ}^{2} \overline{μ} + \frac{{K}_{ma x}^{2}}{μ} + \frac{κ {K}_{ma x}^{3}}{\overline{λ}} ) ( log \frac{2 {t}_{m ix}}{δ} + 8 ( {d}_{S} + {d}_{A} ) ) ,$$

then with probability 1−δ, one has

$${‖ P - \hat{P} ‖}_{σ} \leq 256 {‖ {Σ}^{- 1} ‖}_{σ} \sqrt{\frac{\overline{λ} ( log \frac{2 {t}_{m i x}}{δ} + {d}_{S} + {d}_{A} ) ( κ + \overline{μ} {‖ {Σ}^{- 1} ‖}_{σ}^{2} )}{( n / {t}_{mix} ) {( log \frac{n}{{t}_{mix}} )}^{- 2}}} .$$

The derivation of P also provides a tractable way to estimate E[φ(s)|s, a] by E[ϕ(s′)|s,a] by Ê[ϕ(s′)|s,a]:=\hat{P}×₁ϕ(s)×₂ψ(a). And one has the following guarantee on the estimation error:

∥ [symbol] [ϕ(s′)|s,a]− [symbol] [ϕ(s′)|s,a]∥≤Kₘₐₓ∥\hat{P}−P∥_{σ}.

Learning State and Action Embeddings

Next, it can be shown how to embed states and actions to low-dimensional representations to be maximally “predictative.” Consider a kernelized diffusion distance of the MDP, which measures similarity in terms of future dynamics restricted to a function class:

$$dist [ ( {s}_{1} , {a}_{1} ) , ( {s}_{2} , {a}_{2} ) ] = {sup}_{{‖ f ‖}_{{ℋ}_{S}} \leq 1} \vert 𝔼 [ f ( {s}^{′} ) \vert {s}_{1} , {a}_{1} ] - 𝔼 [ f ( {s}^{′} ) \vert {s}_{2} , {a}_{2} ] \vert .$$

This distance quantifies how well one can generalize the predicted value at a seen state-action pair (s, a) to a new (s′, a′).

Under the low-tensor-rank assumption, we have P=C×₁U₁^{τ}×₂U₂^{τ}×₃U₃^{τ}, where U₁, U₂, U₃ are columnwisely orthogonal matrices. Then we can define the kernelized state diffusion map, kernelized action diffusion map and their joint map as

ƒ(⋅):=U₁^{τ}ϕ(⋅),g(⋅):=U₂^{τ}ψ(⋅),

Φ(s,a):=C×₁ƒ(s)×₂g(a).

It follows that

dist[(s,a),(s′,a′)]=∥Φ(s,a)−Φ(s′,a′)∥,

if φ is a collection of unit orthogonal basis functions of H_{S}.

One can estimate state and action embedding maps based on the tensor estimator. After obtaining \hat{P}, one can simply find the corresponding state and action embedding maps from factors of its Tucker decomposition

\hat{P}=Ĉ×₁Û₁^{τ}×₂Û₂^{τ}×₃Û₃^{τ},

where we require that Ûₖ, k=1, 2, 3 be column-wisely orthogonal. The full procedure is given in Algorithm 1.

Algorithm 1 Learning State and Action Embedding Maps

| | |
|---|---|
| 1: | Input: {(sᵢ, aᵢ, sᵢ′)}ᵢ₌₁ⁿ, (r, l, m) |
| 2: | $Calculate \overline{F} = \frac{1}{n} {\sum }_{i = 1}^{n} \frac{η ( {a}_{i} )}{π ( {a}_{i} \vert {s}_{i} )} φ ( {s}_{i} ) ∘ ψ ( {a}_{i} ) ∘ φ ( {s}_{i}^{′} )$ |
| 3: | Get \hat{F} as the low-rank approximation of F̅ using (2) |
| 4: | $Calculate \hat{Σ} = \frac{1}{n} {\sum }_{i = 1}^{n} φ ( {s}_{i} ) {φ}^{T} ( {s}_{i} ) , \hat{P} = \hat{F} \times {}_{1}^{ }{\hat{Σ}}{}_{ }^{- 1}$ |
| 5: | Let P₁ = \hat{P}. For each k = 1, 2, 3, derive Uₖ from the SVD |
| | of [symbol] ₖ (Pₖ): |
| | [symbol] ₖ (Pₖ) = UₖΛₖVₖ^{T} |
| | and let Pₖ₊₁ = Pₖ × ₖUₖ. |
| 6: | Output: |
| | State embedding map \hat{f} : s [symbol] Û₁^{T} ϕ(s); |
| | Action embedding map ĝ : a [symbol] Û₂^{T} ψ(a); |
| | Core transition tensor Ĉ = P₄. |

Now one has obtained the state embedding map f and the action embedding map ĝ. Accordingly, one can define the joint state-action embedding as

\hat{Φ}(s,a)=Ĉ×₁\hat{ƒ}(s′)×₂ĝ(a′),

and define the empirical embedding distance as

[symbol]

[s,a),(s′,a′)]=∥\hat{Φ}(s,a)−\hat{Φ}(s′,a′)∥.

The following theorem shows that the learned embeddings preserve the kernelized diffusion distance dist[⋅,⋅] with an additive error bound.

Theorem 3 (Embedding error bound). Let Assumptions 1-2 hold. Suppose ϕis an orthogonal basis of H_{S}, and ψ is orthogonal with respect to L²(η), then we can find an orthogonal matrix O, such that ∀s, a, s′, a′,

∥\hat{Φ}(s,a)−OΦ(s,a)∥≤ϵ,

| [symbol] [(s,a),(s′,a′)]−dist[(s,a),(s′,a′)]|≤2ϵ,

where ϵ is controlled by the low-rank estimation error

$$ϵ := {K}_{m ax} ( 1 + \sqrt{2} \frac{{‖ P ‖}_{σ}}{σ} ) {‖ \hat{P} - P ‖}_{σ} ,$$

and σ:=sup_{∥w∥≤1}Σₘ(P×₁w), where σₘ denotes the m-th singular value of a matrix.

An approximation guarantee for transition and value functions. In downstream tasks such as in RL, one often needs to use \hat{ƒ}, ĝ, \hat{Φ} for function approximation. Suppose that one needs to evaluate the one-step expected function [symbol] ƒ for some f ∈ H_{S}. Then one can show that the learned state-action representation \hat{Φ}(⋅) is good enough for approximating the function [symbol] ƒ. In particular, there always exists g(⋅) εSpan \hat{Φ}(⋅)) such that ∥g= [symbol] ƒ∥_{H}_{_{S}}≤ϵ∥ƒ∥_{H}_{_{S}}.

Advantage of tensor method. As an alternative, one could treat the state and action jointly and learns a low-dimensional representation for (s, a) directly. This would not require any tensor decomposition, and matrix methods are enough. This approach may be favorable if the (s, a) is known to have a very simple joint structure.

On the other hand, the tensor approach can be significantly more sample efficient if states and actions admit separate low-dimensional structures, especially if one wants to decouple them and find an optimal discretized MDP approximation. To see this, suppose the action features have dimension d_{A} and state features have dimension d_{S} before embedding. Also assume the Tucker rank is r=l=m for simplicity. By treating (s, a) jointly, a matrix decomposition method would require \tilde{Ω}(d_{A}d_{S}r) samples to reliably recover the low-rank structure. In comparison, this tensor-based approach requires only \tilde{Ω} ((dₓ+d_{A})r) samples. Thus, the tensor embedding method is much more sample efficient for decoupling states and actions.

Estimating the Optimal Discrete MDP Abstraction

One can provably reduce a continuous-state continuous-action MDP into a discrete one, by a direct application of the learned kernelized diffusion distance to partition the state and action spaces.

Optimal Partition of State and Action Spaces

The goal is to learn an optimal discretization of a continuous MDP. Specifically, one wants to find a partition of S and A, denoted as blocks Aᵢ, Bⱼ, i∈[nₛ], j∈[nₐ] and a collection of probability transition distributions {qᵢⱼ(⋅)} on the blocks. For each state-action pair (s,a) ∈Aᵢ×Bⱼ, and some function f∈Hₛ, one wants to approximate the one-step expected value ( [symbol] ƒ)(s,a)=∫p(s′|s,a)ƒ(s′)ds′ by

( [symbol] ƒ)(s,a)=∫p(s′|s,a)ƒ(s′)ds′≈∫qᵢⱼ(s′)ƒ(s′)ds.

One can formalize the optimal state-action partition problem as:

$${min}_{\{ {A}_{i} , {B}_{j} , {q}_{ij} \}} {\sum }_{i , j} {\int }_{{A}_{i} \times {B}_{j}} ξ ( s ) η ( a ) {‖ p ( \cdot \vert s , a ) - {q}_{ij} ( \cdot ) ‖}_{{ℋ}_{S}}^{2} dsda , \qquad ( 3 )$$

whose solution is denoted as {Aᵢ*, Bᵢ*, qᵢⱼ*}, and the corresponding optimal value is denoted by L*.

Lemma 5. Given the optimal partition {Aᵢ*, Bᵢ*}, the block-to-state distribution

$${q}_{ij}^{*} ( \cdot ) = \frac{1}{ξ ( {A}_{i}^{*} ) η ( {B}_{j}^{*} )} {\int }_{{A}_{i}^{*} \times {B}_{j}^{*}} ξ ( s ) η ( a ) p ( \cdot \vert s , a ) dsda .$$

is an optimal solution to (3).

In particular, if |A|=1, the MDP reduces to a Markov process. If this Markov process is reversible, the optimization problem reduces to min_{A}_{ᵢ}min_{{q}_{ᵢ}_{}}Σᵢ₌₁ⁿ^{ₛ}∫(s)∥p(⋅|s)−qᵢ(⋅)∥_{H}_{_{S}}²dsda, which becomes equivalent to the metastable state partition problem for random walk and dynamic systems.

Decoupled State and Action Clustering

Next, consider the RL setting where one wishes to learn {Aᵢ*, Bⱼ*} even though p is unknown. Observe that the optimal partition is determined solely by the kernelized diffusion distance equipped by the state-action space. This allows the approximation of (3) by the empirical state-action clustering problem:

$${min}_{\{ {A}_{i} , {B}_{j} , {z}_{ij} \}} {\sum }_{i , j} {\int }_{{A}_{i} \times {B}_{i}} ξ ( s ) η ( a ) \cdot {‖ \hat{Φ} ( s , a ) - {z}_{ij} ‖}^{2} dsda , \qquad ( 4 )$$

whose solution is denoted by {Âᵢ,\hat{B}ᵢ,\hat{z}ᵢ}. Then the corresponding discrete approximation has the following transition distribution from state abstraction i and action abstraction j:

\hat{q}ᵢⱼ(⋅)=\hat{z}ᵢⱼ^{τ}Û₃^{τ}ϕ(⋅).

To facilitate computation, one can provide a relaxation of problem (4) that can be solved easily using k-means-type algorithms. By taking zᵢⱼ=Ĉ×₁ƒᵢ×₂gⱼ for some fᵢ, gⱼ, the partition problem becomes

$${min}_{\{ {A}_{i} , {B}_{j} \}} {min}_{\{ {f}_{i} , {g}_{j} \}} {\sum }_{i , j} {\int }_{{A}_{i} \times {b}_{j}} ξ ( s ) η ( a ) \cdot {‖ \hat{C} {\times }_{1} \hat{f} ( s ) {\times }_{2} \hat{g} ( a ) - \hat{C} {\times }_{1} {f}_{i} {\times }_{2} {g}_{j} ‖}^{2} dsda .$$

Using the relation

∥Ĉ×₁\hat{ƒ}(s)×₂ĝ(a)−Ĉ×ₕ₁ƒᵢ×₂gⱼ∥²

≤2∥Ĉ×₁(\hat{ƒ}(s)−ƒᵢ)×₂ĝ(a)+Ĉ×₁ƒᵢ×₂(ĝ(a)−gⱼ)∥²

≤2∥Ĉ∥_{σ}²Kₘₐₓ(∥\hat{ƒ}(s)−ƒᵢ∥²+∥ĝ(a)−gⱼ∥²),

one obtains a relaxation of problem (4) given by

$${min}_{\{ {A}_{i} , {B}_{j} \}} {min}_{\{ {f}_{i} , {g}_{j} \}} {\sum }_{i , j} {\int }_{{A}_{i} \times {B}_{j}} ξ ( s ) η ( a ) \cdot ( {‖ \hat{f} ( s ) - {f}_{i} ‖}^{2} + {‖ \hat{g} ( a ) - {g}_{j} ‖}^{2} ) dsda .$$

It is equivalent to solving two problems separately:

$${min}_{{A}_{i}} {min}_{{f}_{i}} {\sum }_{i} {\int }_{{A}_{i}} ξ ( s ) {‖ \hat{f} ( s ) - {f}_{i} ‖}^{2} ds ;$$

$${min}_{{B}_{j}} {min}_{{g}_{j}} {\sum }_{j} {\int }_{{B}_{j}} η ( a ) {‖ \hat{g} ( a ) - {g}_{j} ‖}^{2} da .$$

In short, one can efficiently compute the decoupled state and action clusters, by using the learned representations from the tensor method. The full procedure is given in Alg. 2.

Algorithm 2 Learning Optimal State-Action Abstractions

| | |
|---|---|
| 1: | Input: {(sᵢ, aᵢ, sᵢ′)}ᵢ₌₁ⁿ, (r, l, m) |
| 2: | Estimate the state embedding and action embedding maps |
| | \hat{f}, ĝ, Û₃ using Algorithm 1. |
| 3: | Apply k-means to solve the following two problems, respectively: |
| | ${min}_{\{ {A}_{i} \}} {min}_{\{ {f}_{i} \}} {\sum }_{i}^{ } {\int }_{{A}_{i}}^{ } ξ ( s ) {‖ \hat{f} ( s ) - {f}_{i} ‖}^{2} ds ,$ |
| | ${min}_{\{ {B}_{j} \}} {min}_{\{ {g}_{j} \}} {\sum }_{j}^{ } {\int }_{{B}_{j}}^{ } η ( a ) {‖ \hat{g} ( a ) - {g}_{j} ‖}^{2} da .$ |
| 4: | Output: |
| | The state partition {Âⱼ} and action partition {\hat{B}ⱼ} |

Using this method, the empirical discretion is not far from the groundtruth transition probability.

Once can test the proposed approach on a particular MDP derived from a controlled stochastic process. Set the state and action spaces to be R¹ and R². At each step k, suppose the current state-action pair is (sₖ, aₖ). The next state sₖ₊₁is set to be X_{τ(k+1)} for some τ>0, where Xₜ is the solution of the following SDE:

dXₜ=−[∇V(Xₜ)+F(aₖ)]dt+\sqrt{2}dBₜ,kτ≤t≤(k+1)τ.

Let V (⋅) be a wavy potential function and let F (⋅) be a block-wise constant function. Let Bₜ be the standard Brownian motion. Let the behavior policy be always choosing a from a standard normal distribution. Also let η be a 2-d standard normal distribution. One can use the Gaussian kernels

$${K}_{1} ( x , y ) = {K}_{2} ( x , y ) = \frac{1}{2 π {σ}^{2}} {e}^{- \frac{{‖ x - y ‖}^{2}}{2 {σ}^{2}}}$$

and obtain the state features ϕ (or action features ψ) by first generating (e.g., N=2000) random Fourier features h=[h₁, h₂, . . . , h_{N}] such that K₁(x,y)≈Σᵢ₌₁^{N} hᵢ(x)hᵢ(y) and then orthogonalizing them with respect to L²(ξ) (or L²(η)) to get ϕ (or ψ). Then, one can use τ=0.1, σ=0.5,(r,l,m)=(4,3,4) and the sample size n=10⁶, and apply Algorithm 2 to estimate state and action clusters. Comparing them with the groundtruth, one can validate that this method indeed reveals the latent state and action blocks.

### Example 3

In some embodiments of the method and system, the method and/or system can be used to evaluate and recommend physicians, if the actions are viewed as choosing physicians. For example, a hospital could have collected many clinical episodes from multiple physicians. The algorithm can estimate the expected cost/value of a physician for his contribution to any individual segment/portion of the episode. Using this approach, the method and/or system can predict the clinical pathway for any physician, or for a subgroup of physicians. Such information makes it possible for the healthcare providers to decompose the complicated clinical process and identity their strengths/weakness in each step of this process.

### Example 4

In some embodiments of the method and system, the method and/or system are be used to predict the performance (for example, cost and healthcare outcome) of a new treatment policy that has never been implemented before, based on existing data generated by following current treatment policies. The method can also be used to compute tight statistical confidence interval for the predictions. It can be mathematically proven that the proposed method is optimal in the statistical sense for such task. See, e.g., Duan Y, Wang M. Minimax-Optimal Off-Policy Evaluation with Linear Function Approximation. arXiv preprint arXiv:2002.09516. 2020 Feb. 21, which is incorporated by reference herein in its entirety.

Those skilled in the art will recognize or be able to ascertain using no more than routine experimentation many equivalents to the specific embodiments of the invention described herein. Such equivalents are intended to be encompassed by the following claims.

What is claimed:

1. A system for providing health care providers with optional best-practice care options, comprising:
a. one or more processors configured to extract features in a low-dimensional space in order to identify one or more policies, by:
i. modeling a decision-making process as a dynamic state-transition process comprising a series of states and actions, and generate a kernel function that captures the similarity between any two states, which is an approximate divergence function between their future distributions of pathways, based on applying a state embedding learning technique to sequential clinical records;
ii. determining an empirical parametrized or non-parametrized transition model and an empirical reward function based on the sequential clinical records and providing statistical confidence bounds for the estimated transition model and its predictions;
iii. generating a sequence of converging solutions towards an optimal value function and an optimal decision policy that maps the optimal decision for each possible state;
iv. obtaining one or more policies based on at least one of the converging solutions, the one or more policies can be used to prescribe actions sequentially along a series of states;
v. using either one or more the optimized policies obtained in the previous step or any user-specified policy to compute and predict at least one probabilistic future clinical pathway that a patient would go through conditioned on the patient's current records;
vi. using either one or more optimized policies obtained in the previous step or any user-specified policy to compute and predict the posterior probability distribution of two or more most likely clinical pathways that a patient would go through conditioned on the patient's current records;
vii. using either one or more optimized policies obtained in the previous step or any user-specified policy to compute and predict the overall financial cost or other specified outcome and the estimated value's statistical confidence region;
b. one or more remote computing devices capable of communicating with the one or more processors, the one or more remote computing devices configured to:
i. receive input from a user relating to a single health-related episode;
ii. transmit the input to the one or more processors; and
iii. graphically display the one or more policies relating to the health-related episode.

2. The system according to claim 1, further comprising a database containing the plurality of sequential clinical records.

3. The system according to claim 1, wherein the one or more processors is configured to receive the input, parse the input, and add a record to the database.

4. The system according to claim 1, wherein the one or more policies includes a policy having the lowest overall cost or a policy where each individual claim cost in the policy is below a predetermined threshold.

5. The system according to claim 1, wherein each record comprises a plurality of attributes, the attributes including a cost, and a code representing a diagnosis, a procedure code, at least one start date, and at least one end date.

6. The system according to claim 1, wherein the input consists of health-related information of a single patient.

7. The system according to claim 1, wherein the input consists essentially of a diagnosis.

8. The system according to claim 1, wherein the one or more processors are configured with automatic feature generation and feature selection based on the techniques of state aggregation learning and state embedding learning, and tensor decomposition-based state-action representation learning.

9. The system according to claim 1, wherein the one or more processors are further configured to predict a financial cost for a current treatment, a financial risk for one or more future treatments, number of future hospital visits, hospitalization duration, health condition at the end of episode, likelihood of future complications, other user-specified outcome or a combination thereof.

10. The system according to claim 1, wherein the one or more processors are further configured to estimate leading features and a statistically-optimal reduced-dimension model of state-transition process from trajectorical data via unsupervised spectral state compression.

11. The system according to claim 1, wherein each state is modeled as sₜ=(most recent and/or all related records for a patient up to time t, number of times the patient has been inpatient up to time t).

12. A method for determining optimal policies for a health-related process, comprising:
a. receiving input relating to a single health-related episode;
b. obtaining one or more policies based on at least one converging solution, the one or more policies can be used to prescribe actions sequentially along a series of states;
c. using the one or more policies to compute and predict at least one probabilistic future clinical pathway that a patient would go through conditioned on the patient's current records;
d. transmitting the one or more policies to a remote computer, each policy indicating potential actions to be taken currently or in the future; and
e. graphically or numerically displaying the one or more policies to a user of the remote computer.

13. The method according to claim 12, further comprising:
a. determining an empirical transition model and an empirical reward function based on the sequential clinical records and provide statistical confidence bounds for the empirical transition model and its predictions, using a trained model that is optimized to model a decision-making process as a dynamic state-transition process comprising a series of states, and includes a kernel function that has been generated to capture the similarity between any two states, based on a database containing sequential clinical records;
b. generating a sequence of converging solutions towards an optimal value function;
c. calculate one or more policies based on at least one of the sequence of converging solutions.

14. The method according to claim 12, further comprising determining a treatment option for a patient based on the displayed one or more policies.

15. The method according to claim 12, wherein the one or more policies are based on an insurance-related factor.

16. The method according to claim 12, further comprising parsing the input and adding a record to the database.

17. The method according to claim 12, wherein each record comprises a plurality of attributes, the attributes including a cost, and a code representing a diagnosis, a procedure code, at least one start date, and at least one end date.

18. The method according to claim 12, wherein the input consists of health-related information of a single patient, or wherein the input consists essentially of a diagnosis.

19. The method according to claim 12, wherein the one or more processors are configured with automatic feature generation and feature selection based on state aggregation learning and state embedding learning.

20. The method according to claim 12, wherein the one or more processors are further configured to predict a financial cost for a current treatment, a financial risk for one or more future treatments, number of future hospital visits, hospitalization duration, health condition at the end of episode, likelihood of complication, or other user-specified outcome, or a combination thereof.
